(19) United States
(12) Patent Application Publication
(10) Pub. No.: US 2008/0214576 A1
(43) Pub. Date: Sep. 4, 2008

(54) Topoisomerase Inhibitors and Prodrugs

(75) Inventors: Mark Matteucci, Portola Valley, CA (US); Jian-Xin Duan, South San Francisco, CA (US); Photon Rao, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals, Inc., Redwood City, CA (US)

(21) Appl. No.: 11/791,197

(22) PCT Filed: Nov. 27, 2005
(86) PCT No.: PCT/US2005/041959

§ 371 Date: Nov. 27, 2007

Related U.S. Application Data
(60) Provisional application No. 60/629,723, filed on Nov. 18, 2004.

(51) Int. Cl. A61K 31/519; C07D 491/147; C07D 221/18; A61P 35/00; A61K 31/4365; A61K 31/437; C07D 495/04

(52) U.S. Cl. 514/257; 544/245; 546/48; 546/61; 546/62; 514/283; 514/284; 514/285

(57) ABSTRACT

Compositions and methods for treating cancer and other hyperproliferatice disease conditions with topoisomerase inhibitors and their prodrugs are disclosed.

### CROSS-REFERENCES TO RELATED APPLICATIONS

This application claims the benefit of U.S. Patent Application No. 60/629,723 filed 18 Nov. 2004, the content of which is incorporated herein by reference.

### BACKGROUND OF THE INVENTION

1. Field of the Invention

The present invention provides compositions and methods for treating cancer and other hyperproliferative disease conditions with topoisomerase inhibitors and their prodrugs, and generally relates to the fields of chemistry, biology, molecular biology, pharmacology, and medicine.

2. Description of Related Art

Enzymes known as topoisomerases introduce swivels in DNA strands, allowing the DNA strands to unwind from their normal coiled configuration and assume a topology favorable for replication. Without such an unwinding mechanism, the DNA could not be replicated, and the cell could not reproduce and proliferate (See Andoh, DNA topoisomerase in cancer therapy, Kluwer Academic, New York, 2003, incorporated herein by reference). Type I topoisomerase, also known as topo I, alters DNA topology by creating a transient single-strand break in the DNA and facilitating single-strand passage through the break (Champoux, Ann. Rev. Biochem., 2001, 70:369-413). Type II topoisomerase, also known as topo II, acts by passing an intact DNA helix through a transient double-stranded break generated in a separate segment (Fortune et al., Prog. Nucleic Acid Res. Mol. Biol., 2000, 64:221-253).

It has been recognized that cell proliferation might be controlled by inhibition of topoisomerase enzymes and that such control might be useful in halting the spread of tumors and related malignancies, ultimately destroying them (see Nelson et al., Proc. Nat. Acad. Sci., USA, 1984, 81:1361). Inhibitors of both topo I and topo II have been developed for cancer therapy. However anti-tumor drugs inhibiting topo II are more numerous than those inhibiting topo I.

Anti-cancer drugs reported to work by inhibiting topo II include Adriamycin (doxorubicin hydrochloride), mitoxantrone (Novantrone™, Serono Inc. and OSI Pharmaceuticals Inc.), etoposide (VP-16™), and acridinyl anisidide (m-AMSA, AMSA, or Amsidyl; Andoh, supra). The major drawback of these drugs is their susceptibility to the multi-drug resistance (MDR) phenotype; cancer cells with this phenotype over-express the membrane bound p-glycoprotein pump (see Sikic et al., Cancer Chemother. Pharmacol., 1997, 40 (Suppl):S13-S19).

There remains a need for topoisomerase inhibitors that can be used to treat cancer, particularly for compounds that are easy to synthesize, less susceptible to resistance, and less toxic to normal cells. The present invention meets these needs and provides novel, cancer-cell specific topoisomerase inhibitors, as summarized below.

### BRIEF SUMMARY OF THE INVENTION

In one aspect, the present invention provides topoisomerase inhibitors having a formula selected from the group consisting of:

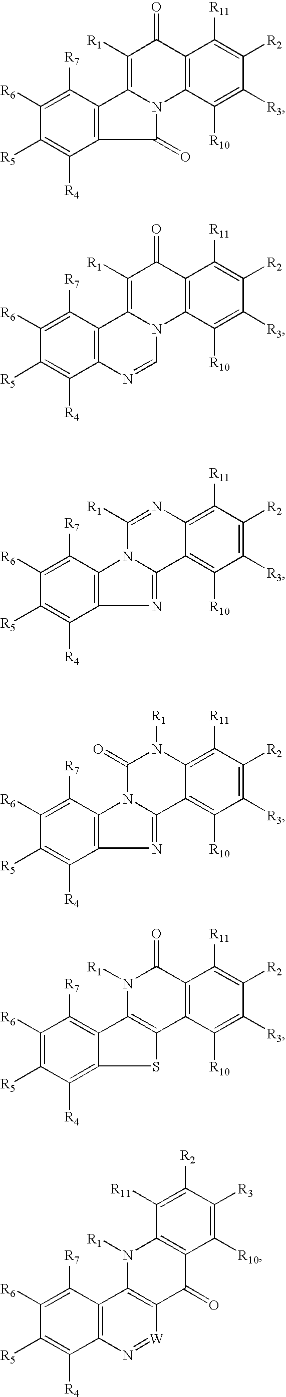

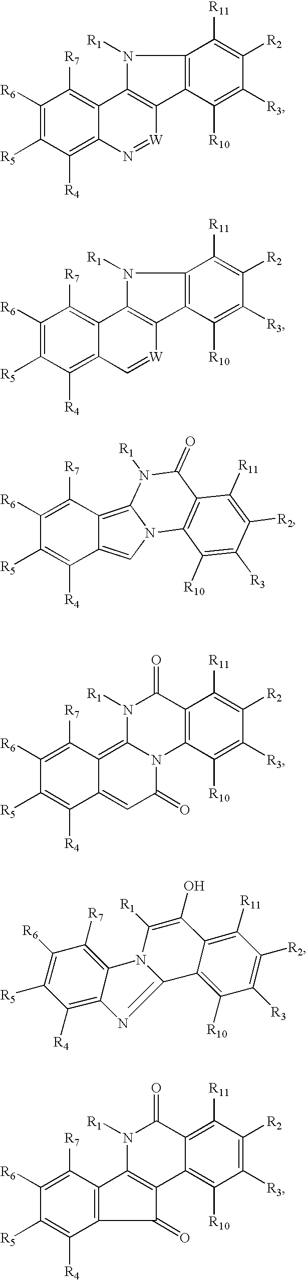

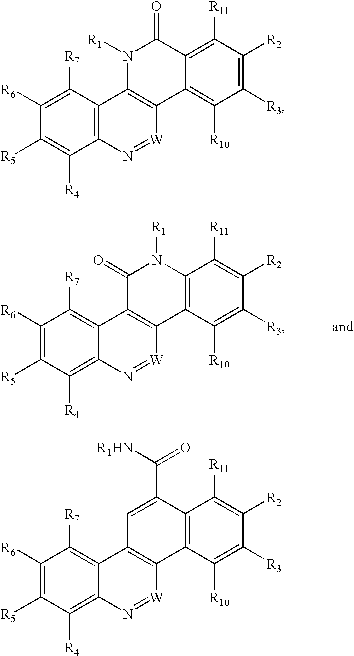

wherein

R₁ is C₁-C₆ alkyl, C₁-C₆ alkoxy, or C₁-C₆ alkylamino;

R₂, R₃, R₁₀, and R₁₁ are independently hydrogen, C₁-C₆ alkoxy, NO₂, amino, aminoalkyl, or hydroxyl;

R₄, R₅, R₆, and R₇ are independently hydrogen, C₁-C₆ alkoxy, NO₂, amino, aminoalkyl or hydroxyl, or R₅ and R₆ together are (—CH₂—O—CH₂—); and

W is —N═ or —CH═;

provided that for:

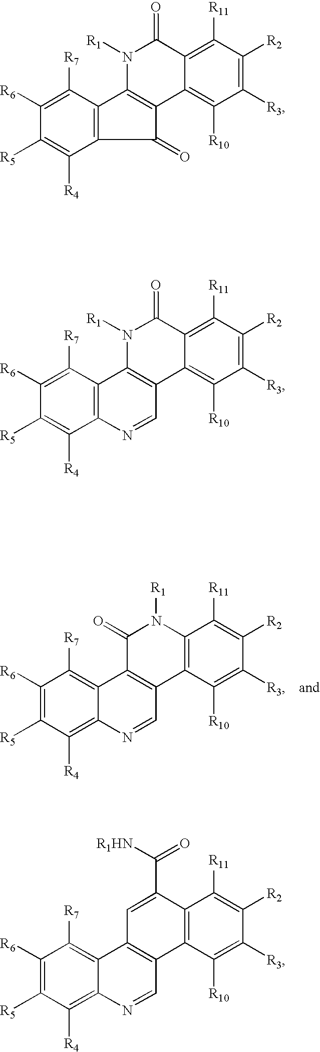

R₁ is not C₁-C₆ alkyl;

provided that for:

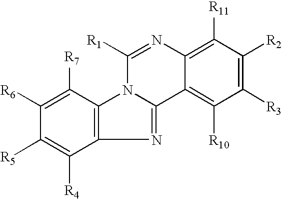

when R₅ and R₆ together are —O—CH₂—O—; R₄, R₇, R₁₀, and R₁₁ are hydrogen; and R₂ and R₃ are —OMe; then R₁ is not —CH₂—CH₂—CH₂—NMe₂; and

provided that for:

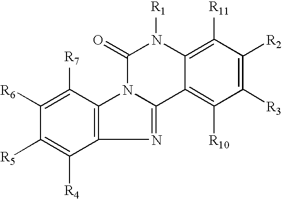

when R₅ and R₆ together are —O—CH₂—O—; R₄, R₇, R₁₀, and R₁₁ are hydrogen; and R₂ and R₃ are —OMe; then R₁ is not —CH₂—CH₂—NMe₂.

In one embodiment, the topoisomerase inhibitors are topo I inhibitors. In one embodiment, the topoisomerase inhibitors are topo II inhibitors.

In another aspect, the present invention provides hypoxia activated prodrugs of topoisomerase inhibitors, including prodrugs of the novel topoisomerase inhibitors of the invention and prodrugs of known topoisomerase inhibitors. In one embodiment, the topoisomerase inhibitors are topo I inhibitors. In one embodiment, the topoisomerase inhibitors are topo II inhibitors.

In another aspect, the present invention provides the following prodrugs of topoisomerase inhibitors:

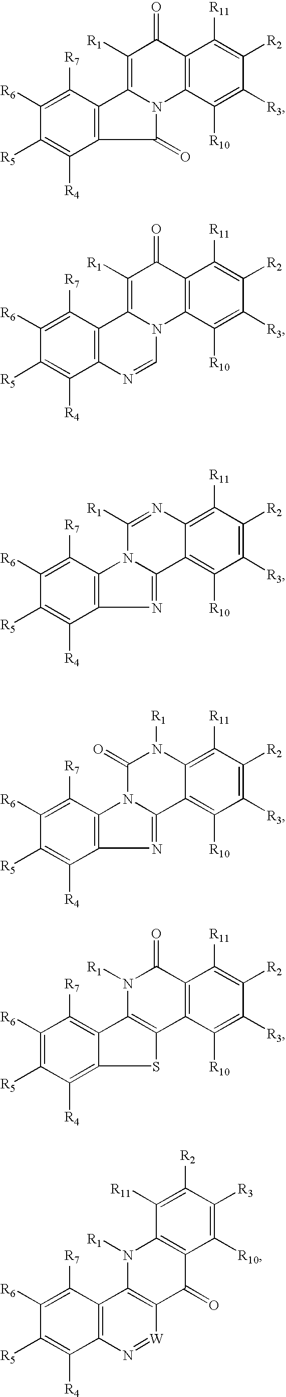

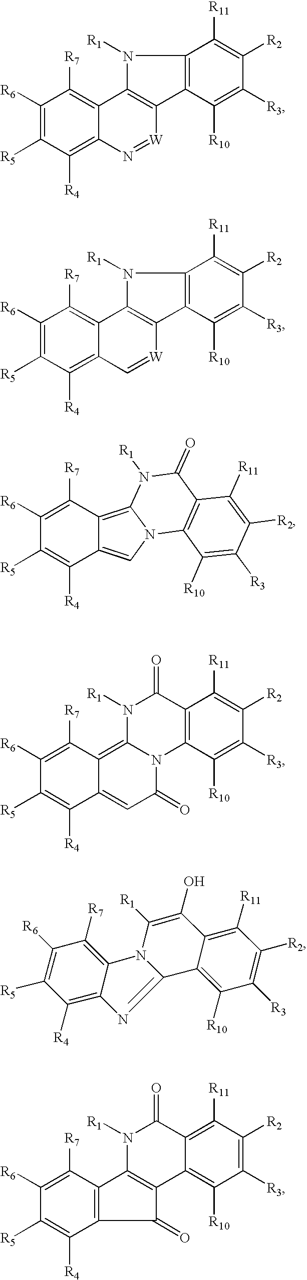

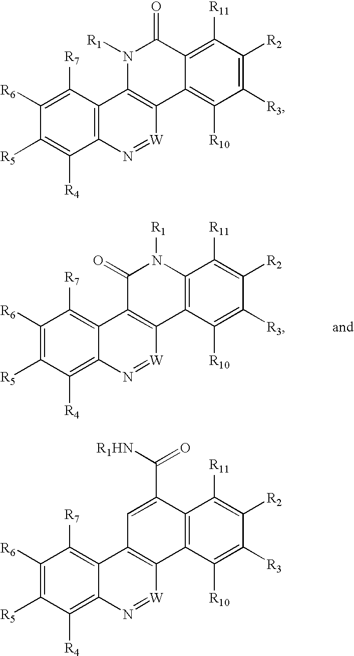

wherein

R₁ is C₁-C₆ alkyl, C₁-C₆ alkoxy, or C₁-C₆ alkylamino,

R₈—[O—(C═O)]ₘ—NR₉—(CH₂)ₙ—, R₈—[O—(C═O)]ₘ—NR₉—(CH₂)ₙ—O—, R₈—[O—(C═O)]ₘ—NR₉—(CH₂)ₙ—NH, R₈—[O—(C═O)]ₘ—NR₉—, or R₈—[O—(C═O)]ₘ—NR₉—(CH₂)ₙ—N(R₈—[O—(C═O)]ₘ)— wherein m is 0 or 1, n is from 1-6, R₈ is a hypoxia labile protecting group, and R₉ is hydrogen or C₁-C₆ alkyl;

R₂, R₃, R₁₀, and R₁₁ are independently hydrogen, C₁-C₆ alkoxy, NO₂, amino, aminoalkyl, hydroxyl, or R₈—[O—(C═O)]ₘ—NR₉— wherein R₈, R₉, m, and n and are defined as above;

R₄, R₅, R₆, and R₇ are independently hydrogen, C₁-C₆ alkoxy, NO₂, amino, aminoalkyl, or hydroxyl, R₈—[O—(C═O)]ₘ—NR₉— wherein R₈, R₉, m, and n are defined as above; or R₅ and R₆ together are (—CH₂—O—CH₂—); and

W is —N═ or —CH═;

provided that if R₁ is C₁-C₆ alkyl, C₁-C₆ alkoxy, or C₁-C₆ alkylamino, then at least one of R₂-R₇, R₁₀, and R₁₁ is R₈—[O—(C═O)]ₘ—NR₉—;

provided that for

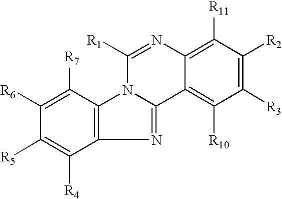

when R₅ and R₆ together are —O—CH₂—O—; R₄, R₇, R₁₀, and R₁₁ are hydrogen; and R₂ and R₃ are —OMe, then R₁ is not —CH₂—CH₂—CH₂—NMe-[(C═O)—O]—R₈; and

provided that for

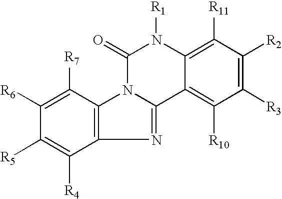

when R₅ and R₆ together are —O—CH₂—O—; R₄, R₇, R₁₀, and R₁₁ are hydrogen; and R₂ and R₃ are —OMe, then R₁ is not —CH₂—CH₂—NMe-[(C═O)—O]—R₈.

In one embodiment, R₈ is selected from:

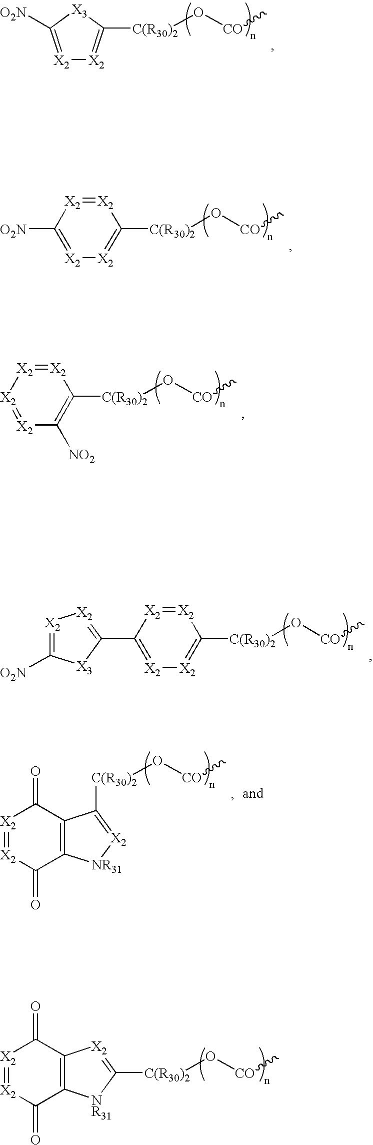

wherein each X₂ is N or CR₃₂;

X₃ is NR₃₁, S, or O;

each R₃₀ is independently hydrogen or alkyl;

R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;

R₃₂ is hydrogen, halogen, nitro, cyano, CO₂H, C₁-C₆alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and

n 0, 1.

In an additional embodiment, R₈ is selected from

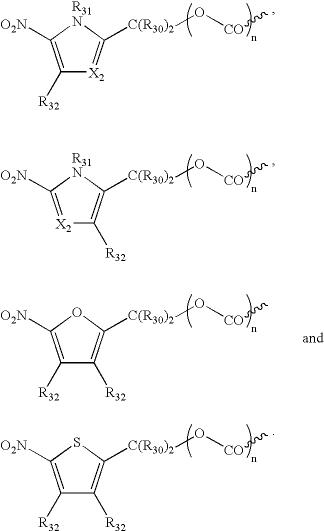

wherein X₂, R₃₀, R₃₁, R₃₂ and n are as defined above.

In another aspect, the present invention provides hypoxia activated prodrugs of topo I inhibitors, including prodrugs of the novel topo I inhibitors of the invention and prodrugs of known topo I inhibitors. In one embodiment, the prodrugs of topo I inhibitors have the structure described above.

In one embodiment, the present invention provides prodrugs of topoisomerase inhibitors comprising at least one hypoxia labile protecting group.

In one embodiment, the present invention provides prodrugs of topoisomerase inhibitors comprising at least two hypoxia labile protecting groups.

In another aspect, the present invention provides pharmaceutical formulations of the topoisomerase inhibitors and the prodrugs of the invention.

In another aspect, the present invention provides methods for making the topoisomerase inhibitors and prodrugs of the invention.

In another aspect, the present invention provides a method for treating cancer in a patient, wherein the method comprises administering to the patient a therapeutically effective amount of a topoisomerase inhibitor or prodrug of the present invention, alone or in combination with one or more other anti-cancer agents.

These and other aspects and embodiments of the invention are described in more detail in the following section.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed to represent a substantial difference over the definition of the term as generally understood in the art.

As used herein, “C₁-C₆ alkyl” or (C₁-C₆) alkyl refers to substituted or unsubstituted straight or branched chain alkyl groups having 1-6 carbon atoms such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl and 3-methylpentyl. A C₁-C₆ alkyl substituent may be covalently bonded to an atom within a molecule of interest via any chemically suitable portion of the C₁-C₆ alkyl group. “C₁-C₆ alkyl” or (C₁-C₆) alkyl may be further substituted with substituents, including for example, hydroxyl, amino, mono or di(C₁-C₆)alkyl amino, halogen, C₂-C₆ alkyl ether, cyano, nitro, ethenyl, ethynyl, C₁-C₆ alkoxy, C₁-C₆ alkylthio, —COOH, —CONH₂, mono- or di-(C₁-C₆)alkylcarboxamido, —SO₂NH₂, —OSO₂—(C₁-C₆)alkyl, mono or di(C₁-C₆)alkylsulfonamido, aryl, and heteroaryl. Substituted C₁-C₆ alkyl groups include, for example, —CH₂—CH₂—OH, —CH₂—CH₂-halogen, —CH₂—CH₂—NH₂, —CH₂—CH₂—O—CH₂—CH₂—OH, —CH₂—CH₂—CH₂—NH—CH₂—CH₂—OH and —CH₂—CH₂—NH—CH₂—CH₂—OH.

As used herein, “C₁-C₆ alkoxy” means a substituted or unsubstituted alkyl group of 1 to 6 carbon atoms covalently bonded to an oxygen atom. A C₁-C₆ alkoxy group has the general structure —O—(C₁-C₆ alkyl) wherein alkyl is as described above. C₁-C₆ alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy.

As used herein, “C₁-C₆ alkoxycarbonyl” refers to an alkoxy group covalently bonded to a carbonyl. A C₁-C₆ alkoxycarbonyl group has the general structure —C(═O)—O—(C₁-C₆)alkyl wherein alkyl is as described above.

As used herein, “C₁-C₆ alkylamino,” means a substituted or unsubstituted alkyl group of 1 to 6 carbon atoms covalently bonded to —NH—. A C₁-C₆ alkylamino group has the general structure —NH—(C₁-C₆)alkyl wherein alkyl is as described above. C₁-C₆ alkylamino groups include, for example, methylamino, ethylamino, propylamino and butylamino.

As used herein, “C₂-C₆ alkyl ether” refers to an ether substituent with 2 to 6 carbon atoms, positioned such that at least one carbon atom is located on either side of the oxygen atom.

As used herein, “aryl” refers to substituted or unsubstituted moieties that include one or more monocyclic or fused ring aromatic systems. Such moieties include any moiety that has one or more monocyclic or bicyclic fused ring aromatic systems, including but not limited to phenyl and naphthyl.

As used herein, “halogen” refers to fluorine, chlorine, bromine, and/or iodine.

As used herein, “heteroaryl” refers to substituted or unsubstituted monocyclic aromatic groups having 5 or 6 ring atoms, or fused ring bicyclic aromatic groups having 8 to 20 atoms, in which the ring atoms are C, O, S, SO, SO₂, or N and at least one of the ring atoms is a heteroatom, i.e., O, S, SO, SO₂, or N. Heteroaryl groups include for example acridinyl, azocinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazole, pyridinyl, pyridyl, pyrimidinyl, pyrrolyl, quinazolinyl, quinolinyl, quinoxalinyl, quinuclidinyl, tetrahydro-isoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl. Unless indicated otherwise, the arrangement of the hetero atoms within the ring may be any arrangement allowed by the bonding characteristics of the constituent ring atoms. Aryl or heteroaryl groups may be further substituted with substituents, including for example, hydroxyl, amino, mono or di(C₁-C₆)alkyl amino, halogen, C₂-C₆ alkyl ether, cyano, nitro, ethenyl, ethynyl, C₁-C₆ alkoxy, C₁-C₆ alkylthio, —COOH, —CONH₂, mono- or di-(C₁-C₆)alkylcarboxamido, —SO₂NH₂, —OSO₂—(C₁-C₆)alkyl, mono or di(C₁-C₆)alkyl-sulfonamido, aryl, and heteroaryl.

As used herein, the term “hydroxy(C₁-C₆)alkyl” refers to a substituted or unsubstituted aliphatic group having from 1 to 6 carbon atoms, and further comprising at least one hydroxyl group on the main carbon chain and/or on a side chain. Hydroxy(C₁-C₆)alkyl groups include, for example, —CH₂—CH₂—OH and —CH₂—CH₂—CH₂—OH.

A wavy line [symbol] indicates the point of attachment of one group or moiety to another.

As used herein, “administering” or “administration of” a drug to a subject (and grammatical equivalents of this phrase) can include both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, an “therapeutically effective amount” of a drug is an amount of a drug that, when administered to a subject with cancer or any other hyperproliferative disease condition, will have (i) the intended therapeutic effect, e.g., alleviation, amelioration, palliation or elimination of one or more manifestations of cancer or other disease in the subject; or (ii) a prophylactic effect, e.g., preventing or delaying the onset (or reoccurrence) of disease or symptoms or reducing the likelihood of the onset (or reoccurrence) of disease or symptoms. The full therapeutic or prophylactic effect does not necessarily occur by administration of one dose and may occur only after administration of a series of doses. Thus, a therapeutically or prophylactically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and can occur only after administration of a series of doses. Thus, a prophylactically effective amount can be administered in one or more administrations.

As used herein, a “second line” therapy is given for a cancer which has failed to respond to a first chemotherapy regimen (called “first line”). “Third line” therapy is that given to a cancer patient when both initial treatment (first-line therapy) and subsequent treatment (second-line therapy) do not work, or stop working is called.

As used herein, a “hypoxia labile protecting group” or “hypoxia activated trigger” refers to a group or moiety that is capable of releasing another compound, such as an antineoplastic agent or analogs thereof, including a topoisomerase inhibitor and analogs thereof, upon hypoxic reduction. In one embodiment, the hypoxia labile protecting group is a group that is capable of releasing the antineoplastic agent or analogs thereof upon reduction of the hypoxia labile protecting group under hypoxic conditions but does not release any antineoplastic agent or analog under normoxic conditions. For example, and as described in more detail below, one hypoxia labile protecting group is a nitroimidazole that may be substituted with a variety of groups. Other examples of hypoxia labile protecting groups include, but are not limited to, groups based on electron deficient nitrobenzenes, electron deficient nitrobenzoic acid amides, nitroazoles, nitroimidazoles, nitrothiophenes, nitrothiazoles, nitrooxazoles, nitrofurans, and nitropyrroles, where each of these classes of moieties may be substituted or unsubstituted, such that the redox potential for the group lies within a range where the group can undergo reduction in the hypoxic regions of a tumor. One of skill in the art will understand, in view of the description herein, how to substitute these and other hypoxia labile protecting groups to provide a redox potential that lies within said range. Additional examples of hypoxia labile protecting group are described in Matteucci et al., PCT Publication No. WO 04/087075 and U.S. Pat. Appl. No. 60/695,755 each of which is incorporated herein by reference.

Generally, one of skill in the art can “tune” the redox potential of a hypoxia labile protecting group by substituting/modifying that group with electron withdrawing groups, electron donating groups, or some combination of such groups. For example, nitrothiophene, nitrofuran, and nitrothiazole groups may be substituted with one or more electron donating groups, including but not limited to methyl, methoxy, or amine groups, to provide a hypoxia labile protecting group with the desired redox potential. In another example, the nitropyrrole moiety can be substituted with an electron withdrawing group, including but not limited to cyano, carboxamide, —CF₃, and sulfonamide groups, to achieve a group with the desired redox potential. For this purpose, strong electron withdrawing groups such as cyano, sulfone, sulfonamide, carboxamide, or —CF₃, and milder electron withdrawing groups such as —CH₂-halogen, where halogen is —F, —Cl, or —Br, can be used.

As used herein, “patient” or “subject” typically refers to a human but more generally refers to a mammal. Those of skill in the art will appreciate that the methods and compositions of the invention can be used to treat cancer or other hyperproliferative diseases in any mammal, including non-human primates, and experimental models of human cancers. In one embodiment, the patient is a human patient.

As used herein, a “pharmaceutically acceptable carrier” includes any and all solvents, dispersion media, coatings, antibacterial and antifungal agents, isotonic agents, absorption delaying agents, and the like, used in the preparation of pharmaceuticals. The use of such media and agents for pharmaceutical active substances is well known in the art. Except insofar as any conventional media or agent is incompatible with the active ingredient, its use in the pharmaceutical formulations of the invention is contemplated. Supplementary active ingredients can be incorporated into the compositions of the invention.

As used herein, a “prodrug” is a compound that, after administration, is metabolized or otherwise converted to an active or more active form with respect to at least one biological property, relative to itself. To produce a prodrug, a pharmaceutically active compound (e.g. a cytotoxic agent) or precursor thereof is modified chemically such that the modified form is less active or inactive, but the chemical modification is effectively reversible under certain biological conditions such that a pharmaceutically active form of the compound is generated by metabolic or other biological processes. A prodrug may have, relative to the drug, altered metabolic stability or transport characteristics, fewer side effects, or lower toxicity (for example, see Nogrady, 1985, Medicinal Chemistry A Biochemical Approach, Oxford University Press, New York, pages 388-392). Those of skill in the art recognize, however, that prodrug synthesis does not necessarily require use of the active drug as synthetic intermediate. Prodrugs can also be prepared using compounds that are not drugs but which upon activation under certain biological conditions generate a pharmaceutically active compound. As used herein a prodrug of a topoisomerase inhibitor is a prodrug that upon activation releases the active topoisomerase inhibitor.

As used herein, “substituent” refers to a molecular moiety that is covalently bonded to an atom within a molecule of interest.

As used herein, the term “substitution” refers to replacing a hydrogen atom in a molecular structure with a substituent such that the valence on the designated atom (for example 4 for carbon) is not exceeded, and a chemically stable compound (a compound that can be isolated, characterized, and/or tested for biological activity) results.

As used herein, “cancer” refers to one of a group of more than 100 diseases caused by the uncontrolled growth and spread of abnormal cells that can take the form of solid tumors, lymphomas, and non-solid cancers such as leukemia.

As used herein, “malignant” refers to cells that have the capacity of metastasis, with loss of both growth and positional control.

As used herein, “neoplasm” (neoplasia) or “tumor” refers to abnormal new cell or tissue growth, which can be benign or malignant.

As used herein, “treating” a condition or patient refers to taking steps to obtain beneficial or desired therapeutic results, including clinical results. Beneficial or desired therapeutic results include, but are not limited to, alleviation or amelioration of one or more symptoms of cancer, diminishment of extent of disease, delay or slowing of disease progression, palliation or stabilization of the disease state, and other beneficial results, as described below.

As used herein, “reduction” of a symptom or symptoms (and grammatical equivalents of this phrase) means decreasing of the severity or frequency of the symptom(s), or elimination of the symptom(s).

As used herein, a “treatment with a topoisomerase inhibitor,” “treatment with a prod rug of a topoisomerase inhibitor,” “anti-neoplastic treatment” “cancer therapy,” “cancer treatment,” or “treatment of cancer,” refers to any approach for ameliorating the symptoms of or delaying the progression of a neoplasm, tumor, or cancer by reducing the number of or growth of cancer cells in the body, typically (but not limited to) by killing or halting the growth and division of cancer cells. As used herein a “drug” refers to a topoismerase inhibitor and analogs and prodrugs thereof, an antineoplastic agent, a cytotoxic agent, a cytostatic agent, and the like.

As used herein a “cytotoxic agent” is an agent that produces a toxic effect on cells. As used herein a “cytostatic agent” is an agent that inhibits or suppresses cellular growth and multiplication.

As used herein “hypoxic cells” are cells residing in a hypoxic environment in vivo such as, for example, in a hypoxic tumor zone, or in vitro. As used herein “normoxic cells” are cells residing in a normoxic environment in vivo or in vitro. As used herein “hypoxic cytotoxicity” of a compound or agent is its cytotoxicity on hypoxic cells. As used herein “normoxic cytotoxicity” of a compound or agent is its cytotoxicity on normoxic cells.

### Compounds

The compounds of the invention can in part be described as topoisomerase inhibitors and prodrugs thereof which prodrugs comprise a hypoxia labile protecting group. In one embodiment, the topoisomerase inhibitors and prodrugs thereof refer to topo I inhibitors and prodrugs thereof, which prodrugs comprise a hypoxia labile protecting group. In one embodiment, the topoisomerase inhibitors and prodrugs thereof refer to topo II inhibitors and prodrugs thereof, which prodrugs comprise a hypoxia labile protecting group. While a number of topo I inhibitory anti-cancer compounds are known, only camptothecin and derivatives thereof have been approved for use in cancer therapy by the FDA. Examples of such approved drugs are irinotecan hydrochloride (Camptosar™, Pfizer) and topotecan hydrochloride (Hycamtin™, Glaxo Smith Kline, see structures below).

These camptothecin analogs have major limitations. At physiological pH the camptothecin analog is in equilibrium with its inactive carboxylate form, which binds to serum albumin (see Burke et al., J. Med. Chem., 1994, 37: 40-46). Additionally the effect of these compounds is reversed within minutes of drug removal, imposing the requirement of long and/or repeated infusions for cancer treatment (see Covey et al., Cancer Res., 1989, 49:5016-5022). Further, development of resistance to such derivatives, attributed to enhanced drug efflux by an ABC transporter, namely the BCRP/MXR/ABCG2 transporter has been reported (see Brangi et al., Cancer Res., 1999, 59:5938-5946; Allen et al., Cancer Res., 1999, 59:4237-41; Maliepaard et al., Cancer Res., 1999, 59:4559-4563; and Schellens et al., Ann. N.Y. Acad. Sci., 2000, 922:188-194, each of which is incorporated herein by reference).

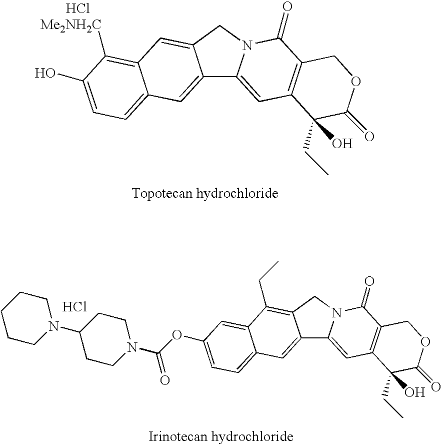

Other non-camptothecin topoisomerase inhibitors are described below.

One class of topo I inhibitors that can kill cancer cells is the indolocarbazole class, which includes rebeccamycin (as illustrated below). See also, Prudhomme et al., Curr. Med. Chem., 2000, 7:1189-1212. Some members of this class, like some camptothecin derivatives, were also found to be eliminated from cancer cells by the BCRP/MXR/ABCG2 transporter (see Komatani et al., Cancer Res., 2001, 61:2827-32).

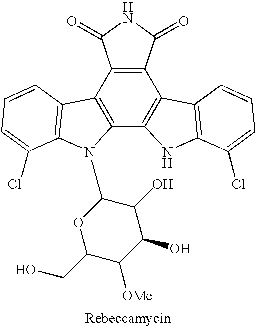

Another class of topo I inhibitors includes benzimidazole derivatives, benzimidazoles (see Kim et al., Bioorg. Med. Chem., 1996, 4; 621), bibenzimidazoles (Kim et al., J. Med. Chem., 1996, 39:992-998), and terbenzimidazoles (Sun et al., J. Med. Chem., 1995, 38:3638-3644). Factors limiting the use of these compounds as anticancer agents include non-specific targeting (Zhang et al., Ann. Clin., Lab. 2001, 31:187-198) and resistance in cell lines over-expressing MDR1 (Chen et al., Cancer Res., 1993, 53:1332-1337).

A number of topo I inhibitory indenoisoquinoline derivatives possessing anticancer activity have been described. (See structure below, Cushman et al., U.S. Pat. No. 6,509,344, and Michalsky et al., U.S. Pat. No. 5,597,831 each of which is incorporated herein by reference). These compounds have a polycyclic core and a substituted alkyl chain joined to the B ring of a polycyclic core as shown below:

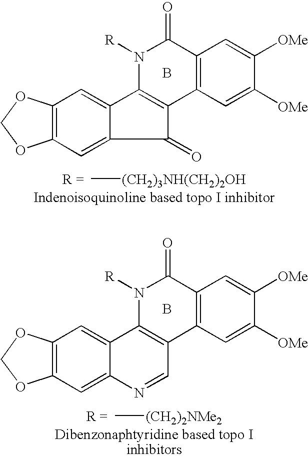

Another class of topo I inhibitory compounds with a polycyclic core is the class of dibenzonaphthyridine analogs (Lavoie et al., PCT Publication No. WO0414916 incorporated herein by reference). Although resistance may be less problematic for the compounds shown above, their synthesis is difficult.

The present invention arises in part out of the discovery that novel topoisomerase inhibitors, including topo I inhibitors, can be designed that are synthesized easily starting from readily available material. As shown in the scheme below, in the compounds of the invention the position of the nitrogen atom on the B ring of the polycyclic core is changed. This change retains the spatial disposition of the molecule, but provides a class of polycyclic compounds (Compound X), that can be easily synthesized from a 4-quinolone compound as an intermediate. Synthesis of a variety of 4-quinolone derivatives useful for other purposes has been described (see, for example, Joseph et al., U.S. Pat. No. 6,645,983 and Iwanowicz et al., US Patent Application Publication No. 2002/0040022, each of which is incorporated herein by reference). Adaptation of these methods in accordance with the present invention provides the synthesis of compounds of the invention (e.g., Compound X) via a 4-quinolone intermediate as described below (see “Methods of Synthesis” for additional details).

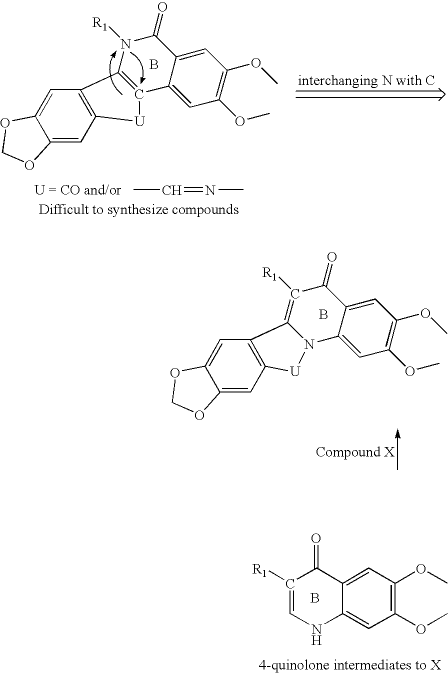

The present invention also provides novel polycyclic topo I inhibitor compounds that are easily synthesized via benzimidazole intermediates as described below.

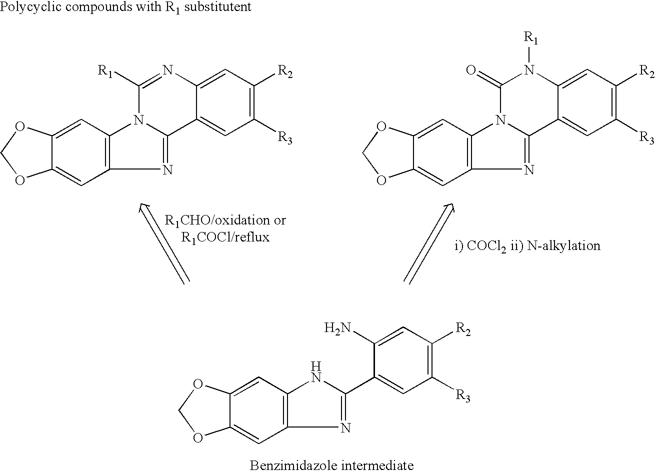

The present invention also provides novel prodrugs of topoisomerase inhibitors, including prodrugs of topo I inhibitors. To understand benefits of these prodrugs, an understanding of tumor biology is helpful. Most drug-mediated cancer therapies rely on cytotoxic agents, selective for dividing cells. These drugs are effective, because cancer cells generally divide more frequently than normal cells. For example, the topo I inhibitors, which are S-phase toxins, target cancer cells, as opposed to normal cells, only to the extent that the former undergo cell division more frequently than normal cells.

However, drugs targeting dividing cells do not always kill all of the cancer cells in a solid tumor. One reason for this lack of complete cell death is that cancer cells can acquire mutations that confer drug resistance. Another reason is that not all cancer cells divide more frequently than normal cells, and slowly-dividing cancer cells can be as, or even more, insensitive to such inhibitors as normal cells. These cells can be slowly-dividing, because they are located in the hypoxic region of the tumor.

As a tumor grows, it requires a blood supply and, consequently, growth of new vasculature. The new vasculature that supports tumor growth is often disordered; leaving significant regions of the tumor under-vascularized and the vascularized regions are also subject to intermittent blockage. Cells in these regions are unable to generate the energy required for cell division. These under-vascularized and blocked regions of the tumor become hypoxic, i.e., they have a lower oxygen concentration than the corresponding normal tissue. Thus, the median oxygen concentration of only ten percent of solid tumors falls in the normal range of 40-60 mm Hg, and fifty percent of solid tumors exhibit median oxygen concentrations of less than 10 mm Hg.

The hypoxic regions of the tumor can constitute a significant reservoir of cancer cells resistant to therapy. Not surprisingly, then, low tumor oxygen levels are associated with a poor response to therapy, increased metastases, and poor survival. In the hypoxic region of a tumor, cancer cells do not divide significantly faster than normal cells, and so can be resistant to therapeutic agents, such as the topo I inhibitors, that target dividing cells.

However, the hypoxic region is conducive to reduction that can be used to generate reduced derivatives of a variety of chemical groups (see Workman et al., 1993, Cancer and Metast. Rev. 12: 73-82), and prodrugs of cytotoxins can be developed to exploit such hypoxic regions (see, Matteucci et al., PCT Application No. US04/009667). Compounds of the present invention arise in part out of the discovery that cancer cells in the hypoxic region can be targeted by prodrugs of topoisomerase inhibitors, wherein the topoisomerase inhibitor has a hypoxia labile protecting group. The hypoxic cells of the tumor generate the active toxin from the inactive, relatively non-toxic prodrug. The active drug diffuses from the hypoxic cells and kills the cancer cells in adjacent regions (where the cells are dividing).

Thus, the hypoxic region acts as a drug-factory to produce a cytotoxin within a tumor for killing adjacent normoxic cancer cells, leading to a higher concentration of the cytotoxin within the tumor, relative to normal tissues. As a result, by employing a prodrug to generate the cytotoxin within the tumor, toxic side-effects arising due to normal cell toxicity can be reduced. After the death of a cancer cell in the normoxic region of the tumor, a hypoxic region can become normoxic and start to divide. At this point, such cells can be killed by the topoisomerase inhibitors generated from the prodrugs of the invention, or by other cytoxins administered in combination with the topoisomerase inhibitors and/or prodrugs of the invention, including topo I inhibitors or other anti-cancer cytotoxins.

Topo I inhibitors with a polycyclic core having an amine functionality can form a cationic ammonium species under physiological conditions. The formation of the ammonium group assists in the formation of a complex comprising topo I inhibitor, DNA, and topo I. The prodrugs of the invention arise in part out of the discovery that conversion of the amine group into a hypoxia labile carbamate can block or inhibit the formation of the ammonium group, which in turn decreases the activity of the topo I inhibitor (see scheme below). Also, conversion of the amine group to a hypoxia labile weakly-basic amine protecting group can decrease ammonium ion formation, leading to inactivation of the toxin.

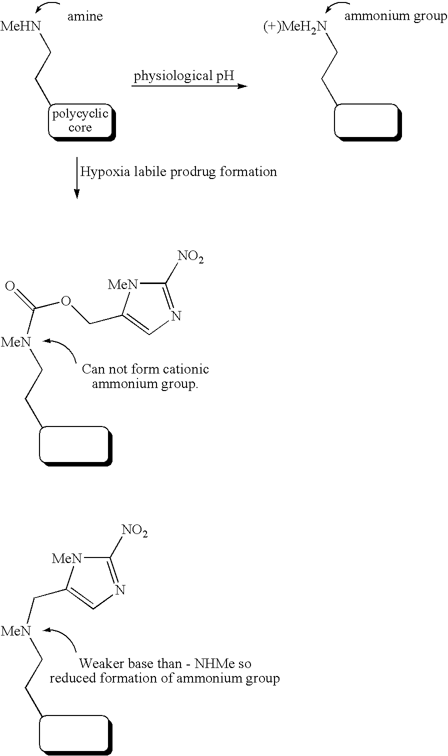

Prodrugs of topo I inhibitors possessing an alkylamino (—NHR) substitution on the polycyclic core can be transformed in accordance with the methods of this invention into a carbamate (—N(CO₂R₄)R) or another amine (—N(CH₂R₄)R; wherein R₄ is a hypoxia labile protecting group. See figure below. Because —CO₂R₄ and —CH₂R₄ groups are both sterically larger than the hydrogen they replace on the alkylamino group, such a substitution can alter the spatial disposition of the —NHR group required in a complex comprising topo I, the topoisomerase inhibitor and DNA for topo I inhibition. Thus, conversion of the alkylamino group (—NHR) of a topo I inhibitor of the invention into a carbamate (—N(CO₂R₄)R) or another amine (—N(CH₂R₄)R) yields an inactive prodrug.

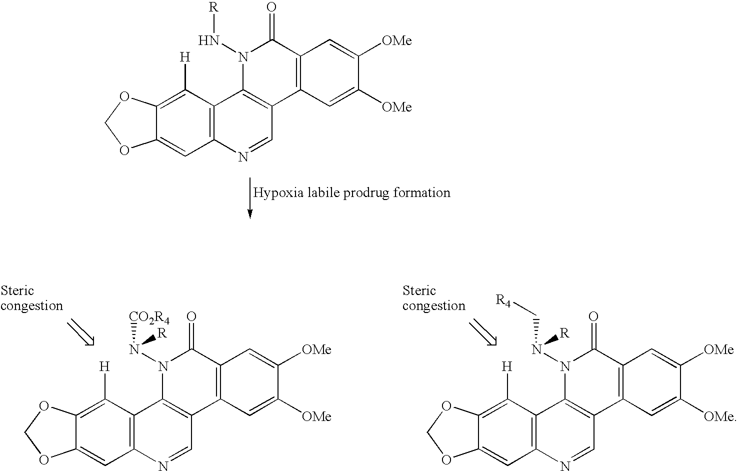

Prodrugs of the present invention can be made in accordance with the methods provided herein by transforming an aromatic amino group of a topo I inhibitor into a carbamate as shown below.

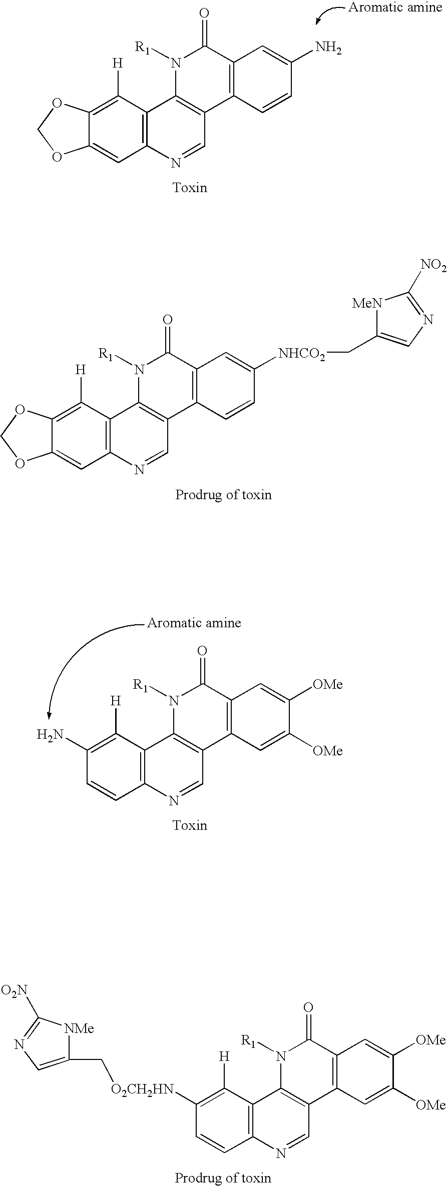

More particularly a compound of the present invention has a structure selected from the group consisting of:

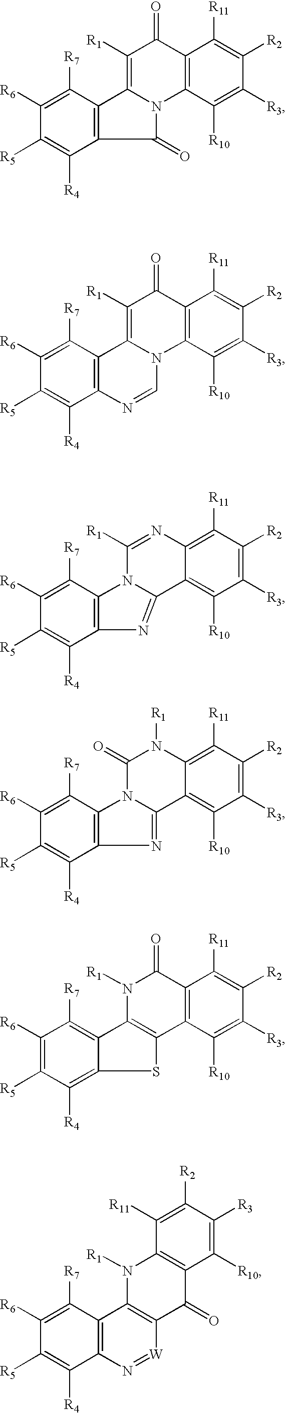

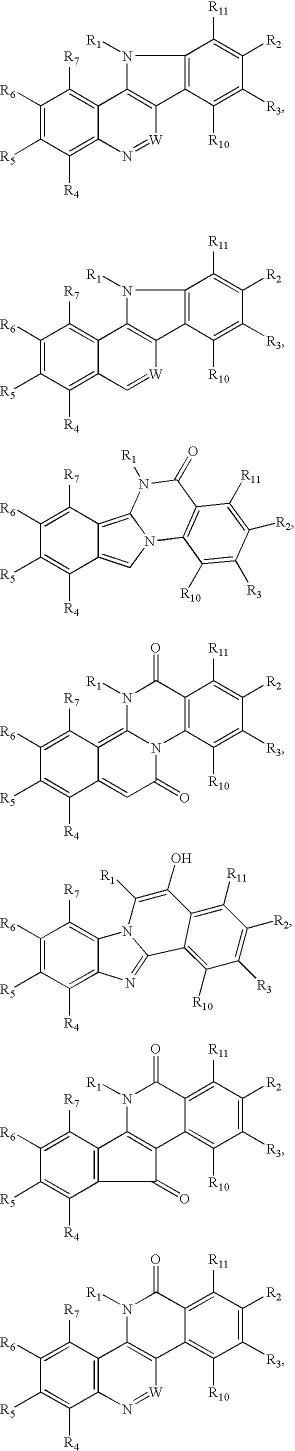

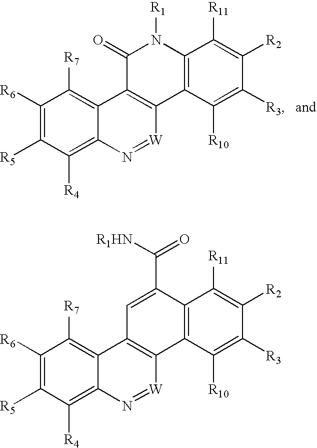

wherein

R₁ is C₁-C₆ alkyl, C₁-C₆ alkoxy, or C₁-C₆ alkylamino;

R₂, R₃, R₁₀, and R₁₁ are independently hydrogen, C₁-C₆ alkoxy, NO₂, amino, aminoalkyl, or hydroxyl;

R₄, R₅, R₆, and R₇ are independently hydrogen, C₁-C₆ alkoxy, NO₂, amino, aminoalkyl, or hydroxyl, or R₅ and R₆ together are (—CH₂—O—CH₂—); and

W is —N═ or —CH═;

provided that for:

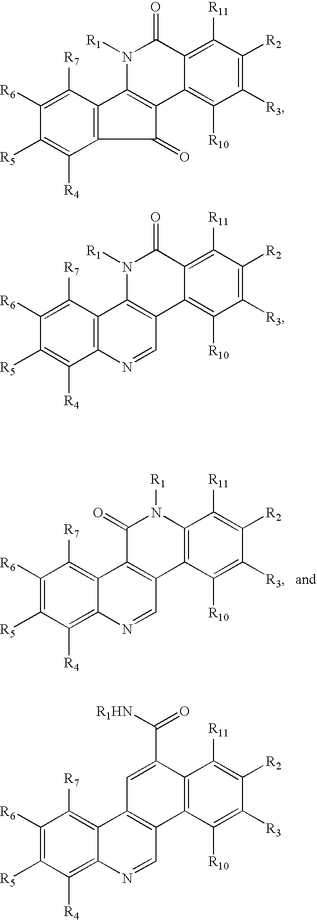

R₁ is not C₁-C₆ alkyl;

provided that for:

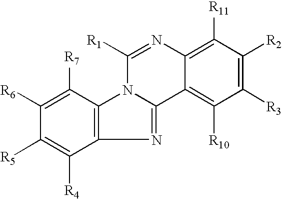

when R₅ and R₆ together are —O—CH₂—O—; R₄, R₇, R₁₀, and R₁₁ are hydrogen; and R₂ and R₃ are —OMe; then R₁ is not —CH₂—CH₂—CH₂—NMe₂; and

provided that for:

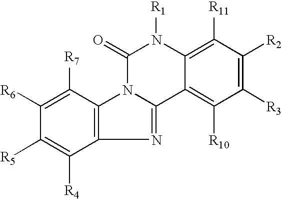

when R₅ and R₆ together are —O—CH₂—O—; R₄, R₇, R₁₀, and R₁₁ are hydrogen; and R₂ and R₃ are —OMe; then R₁ is not —CH₂—CH₂—NMe₂.

In one embodiment, the present invention provides the compounds

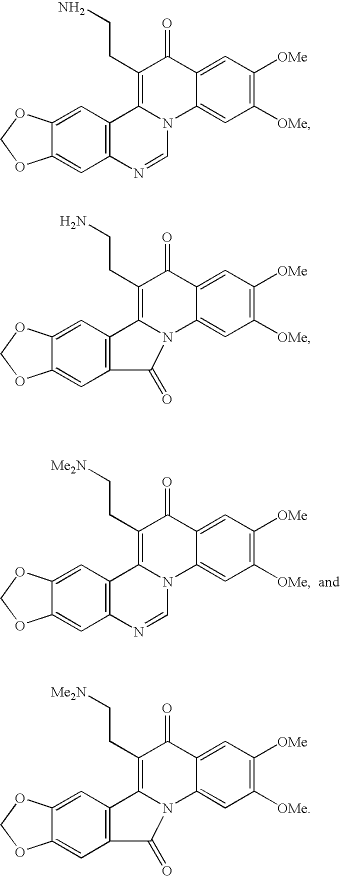

In another aspect, the present invention provides hypoxia activated prodrugs of topoisomerase inhibitors, including prodrugs of the novel topoisomerase inhibitors of the invention and prodrugs of known topoisomerase inhibitors.

In another aspect, the present invention provides hypoxia activated prodrug topo I inhibitors, including prodrugs of the novel topo I inhibitors of the invention and prodrugs of known topo I inhibitors.

In another aspect, the present invention provides the following prodrugs:

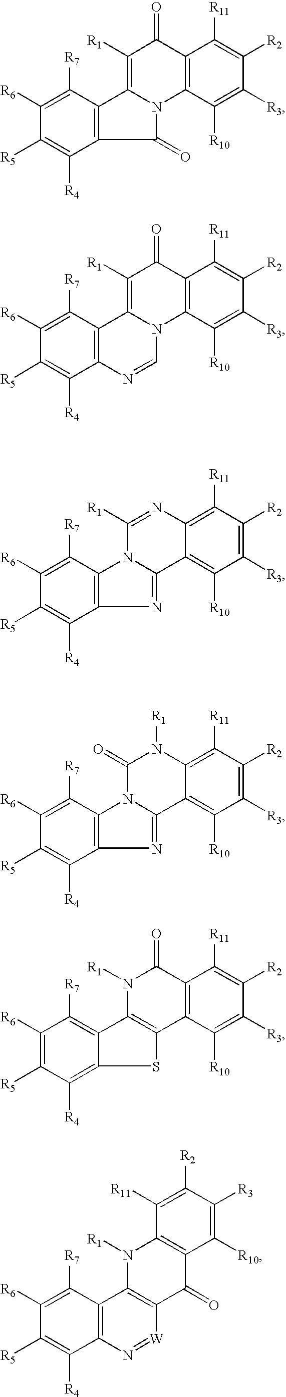

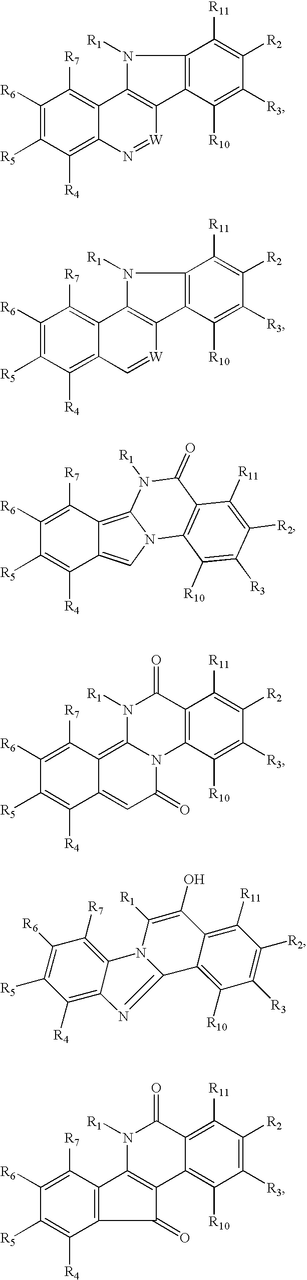

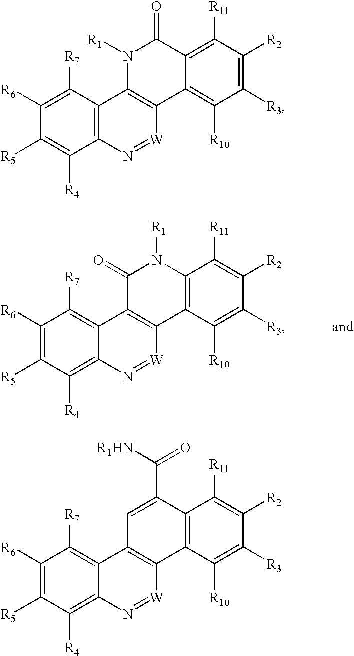

wherein

R₁ is C₁-C₆ alkyl, C₁-C₆ alkoxy, or C₁-C₆ alkylamino, R₈—[O—(C═O)]ₘ—NR₉—(CH₂)ₙ—, R₈—[O—(C═O)]ₘ—NR₉—(CH₂)ₙ—O—, R₈—[O—(C═O)]ₘ—NR₉—(CH₂)ₙ—NH, R₈—[O—(C═O)]ₘ—NR₉—, or R₈—[O—(C═O)]ₘ—NR₉—(CH₂)ₙ—N(R₈—[O—(C═O)]ₘ)— wherein m is 0 or 1, n is from 1-6, R₈ is a hypoxia labile protecting group, and R₉ is hydrogen or C₁-C₆ alkyl;

R₂, R₃, R₁₀, and R₁₁ are independently hydrogen, C₁-C₆ alkoxy, NO₂, amino, aminoalkyl, hydroxyl, or R₈—[O—(C═O)]ₘ—NR₉— wherein R₈, R₉, and m and are defined as above;

R₄, R₅, R₆, and R₇ are independently hydrogen, C₁-C₆ alkoxy, NO₂, amino, aminoalkyl, or hydroxyl, R₈—[O—(C═O)]ₘ—NR₉— wherein R₈, R₉, m, and n are defined as above; or R₅ and R₆ together are (—CH₂—O—CH₂—); and

W is —N═ or —CH═;

provided that if R₁ is C₁-C₆ alkyl, C₁-C₆ alkoxy, or C₁-C₆ alkylamino, then at least one of R₂-R₇, R₁₀, and R₁₁ is R₈—[O—(C═O)]ₘ—NR₉—;

provided that for

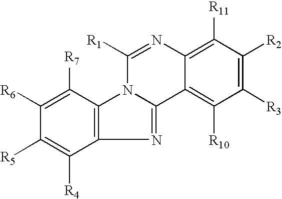

when R₅ and R₆ together are —O—CH₂—O—; R₄, R₇, R₁₀, and R₁₁ are hydrogen; and R₂ and R₃ are —OMe; then R₁ is not —CH₂—CH₂—CH₂—NMe-[(C═O)—O]—R₈; and

provided that for

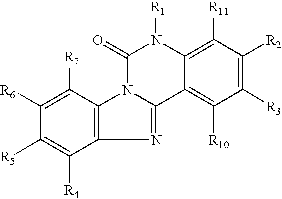

when R₅ and R₆ together are —O—CH₂—O—; R₄, R₇, R₁₀, and R₁₁ are hydrogen; and R₂ and R₃ are —OMe; then R₁ is not —CH₂—CH₂—NMe-[(C═O)—O]—R₈.

In one embodiment, the present invention provides prodrugs comprising at least one hypoxia labile protecting group. In another embodiment, the present invention provides prodrugs comprising one hypoxia labile protecting group.

In one embodiment, the present invention provides prodrugs comprising at least two hypoxia labile protecting groups. In another embodiment, the present invention provides prodrugs comprising two hypoxia labile protecting groups.

In one embodiment, R₈ is selected from:

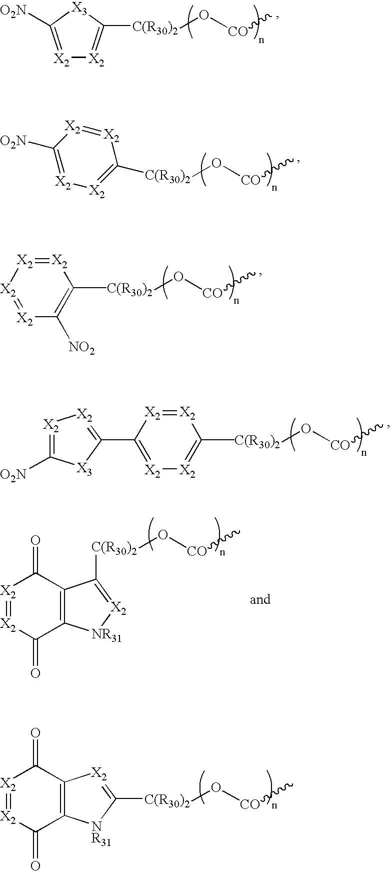

wherein each X₂ is N or CR₃₂;

X₃ is NR₃₁, S, or O;

each R₃₀ is independently hydrogen or alkyl;

R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;

R₃₂ is hydrogen, halogen, nitro, cyano, CO₂H, C₁-C₆ alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and

n=0, 1.

In an additional embodiment, R₈ is selected from

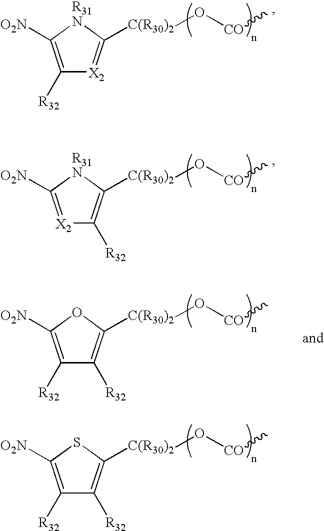

wherein X₂, R₃₀, R₃₁, R₃₂ and n are as defined above.

In one embodiment, the present invention provides the compounds

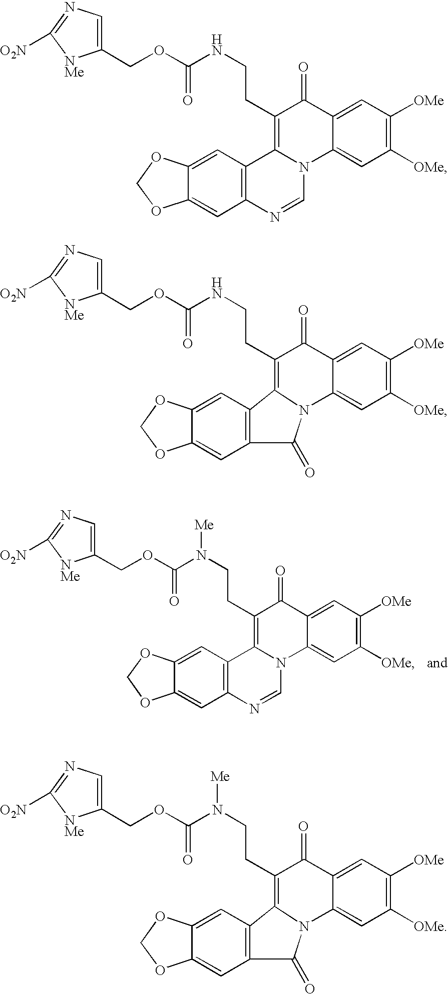

### General Methods of Syntheses

Compounds and prodrugs of this invention can be made by the methods depicted in the reaction schemes shown below.

The starting materials and reagents used in preparing these compounds are either available from commercial suppliers, such as Aldrich Chemical Co., or are prepared by methods known to those skilled in the art following procedures set forth in references such as Fieser and Fieser's Reagents for Organic Synthesis, Wiley & Sons, New York, 1991, Volumes 1-15; Rodd's Chemistry of Carbon Compounds, Elsevier Science Publishers, 1989, Volumes 1-5 and Supplementals; and Organic Reactions, Wiley & Sons, New York, 1991, Volumes 1-40. These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to, filtration, distillation, crystallization, chromatography, and the like. Such materials may be characterized using conventional means, including physical constants and spectral data. Unless specified to the contrary, the reactions described herein take place at atmospheric pressure over a temperature range from about −78° C. to about 150° C., more preferably from about 0° C. to about 125° C.

Methods to synthesize 4-quinolone derivatives are described in U.S. Pat. No. 6,645,983 and US Patent Application Publication No. 2002/040022 (supra). Compounds of the present invention can be synthesized in accordance with this disclosure using as intermediates 4-quinolone derivatives.

The syntheses of polycyclic indenoisoquinoline, dibenzonaphthyridene, and related compounds are described by the references LaVoie et al., Cushman et al. (supra), and Ruchelman et al. Bioorg. Med. Chem. Lett., 2004, 14: 5585-9 (incorporated herein by reference). With these polycyclic compounds used as starting material, prodrugs of the present invention can be synthesized in accordance with the methods provided herein. Prodrugs of novel topoisomerase inhibitors of the invention can be synthesized using reactive steps similar to the above mentioned procedure. In one embodiment, methods for the synthesis of the compounds of the invention can be identified via search tools such as SciFinder from the American Chemical Society and Beilstein from MDL Software. Illustrative methods for making topoisomerase inhibitors and prodrugs of the present invention are also provided schematically below.

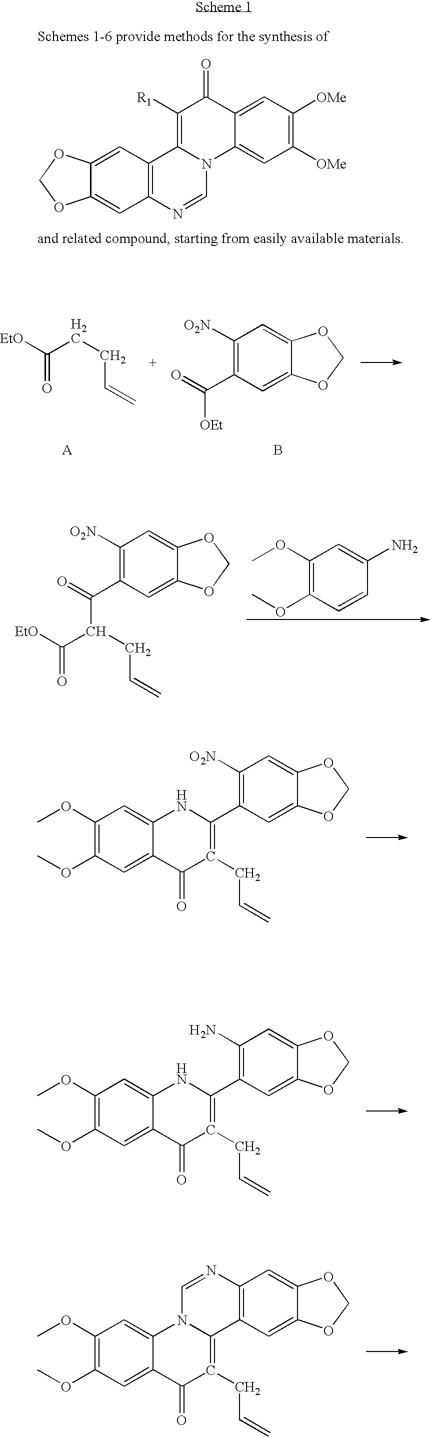

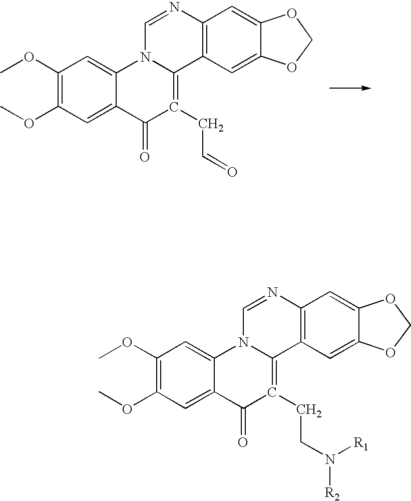

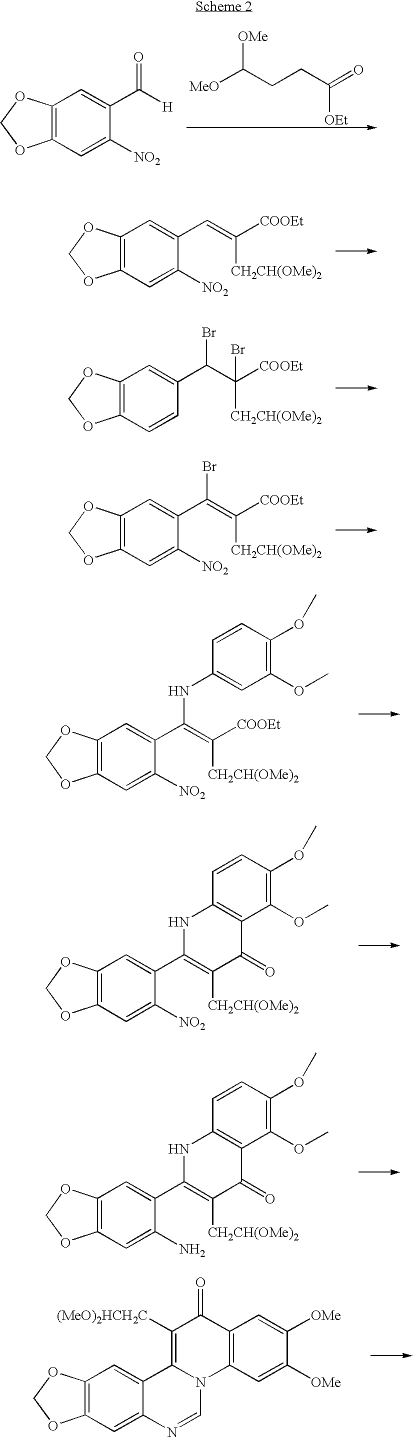

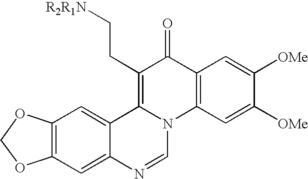

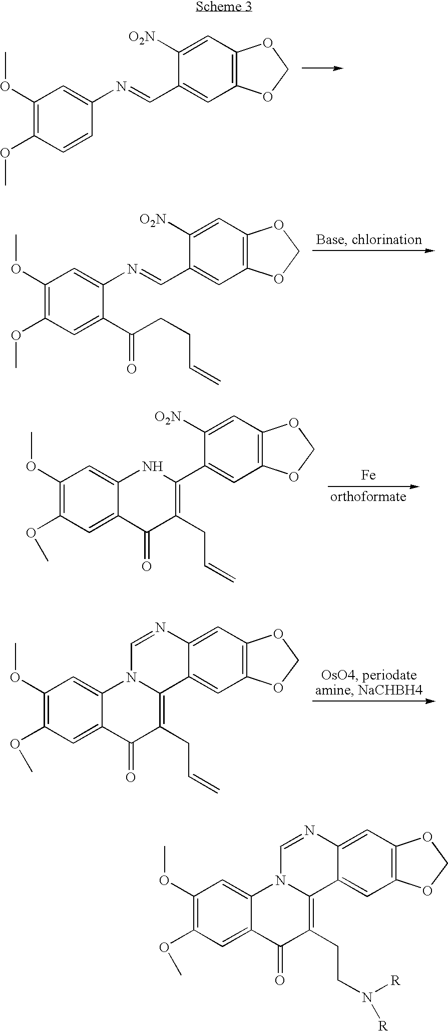

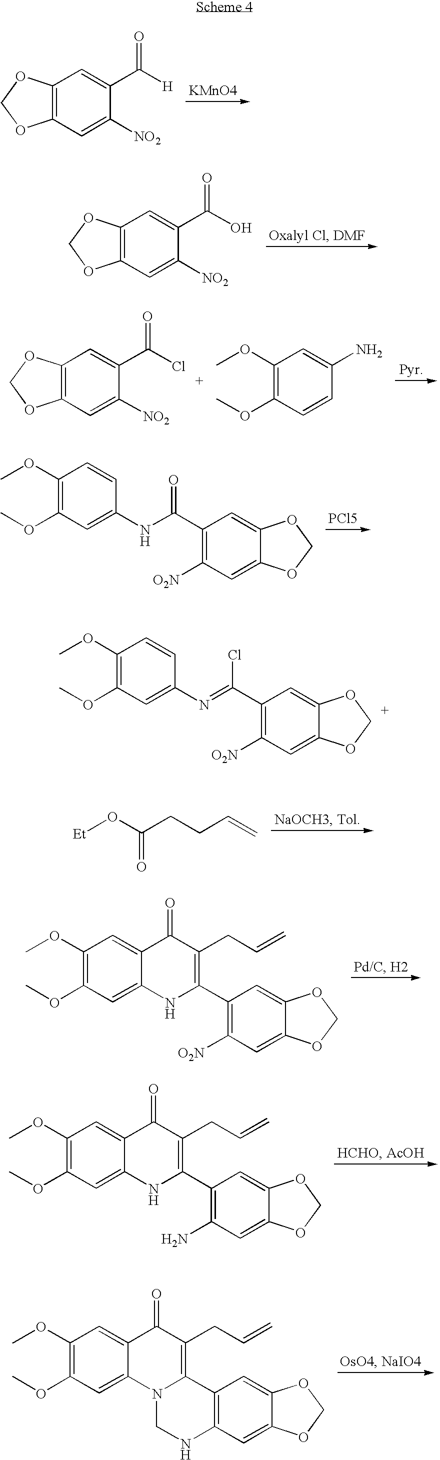

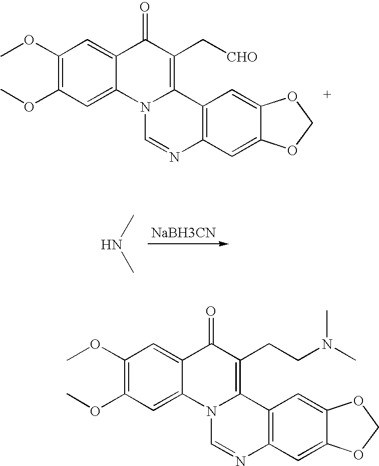

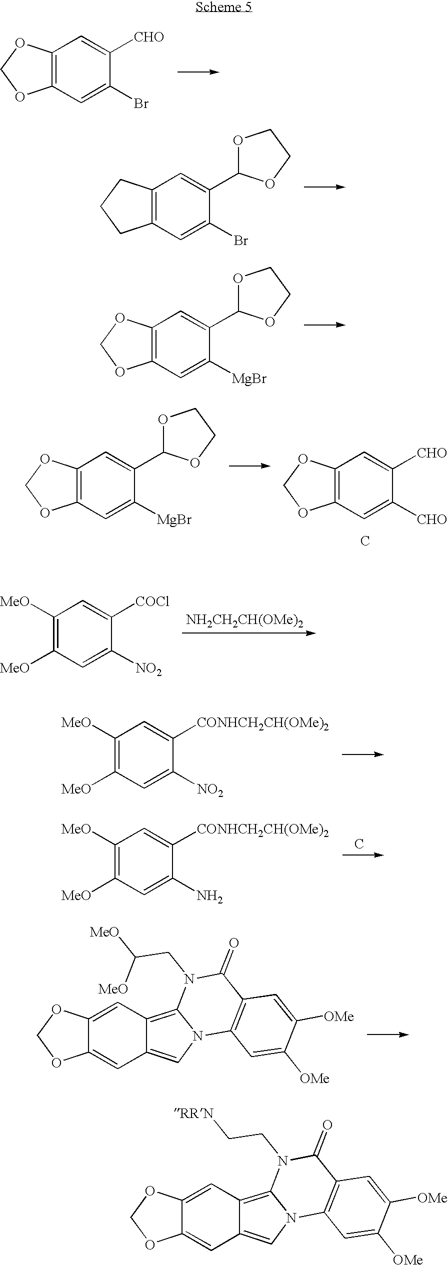

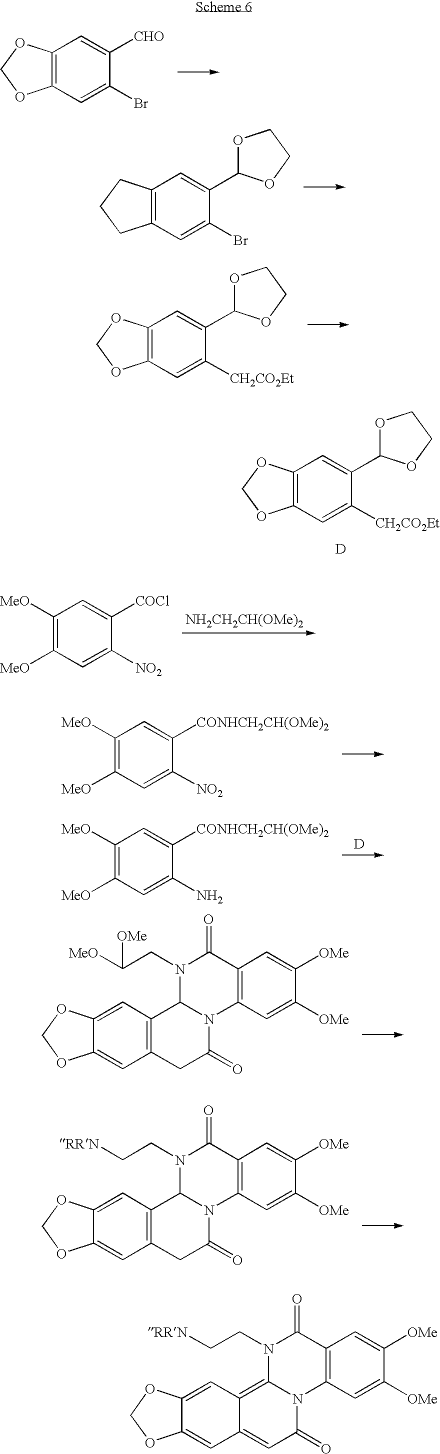

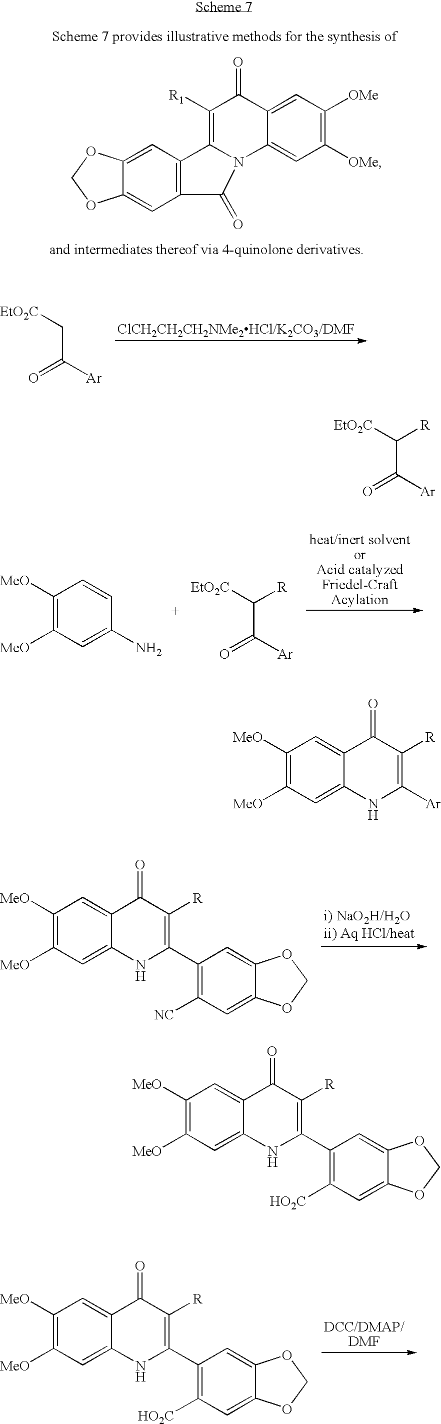

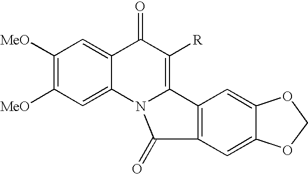

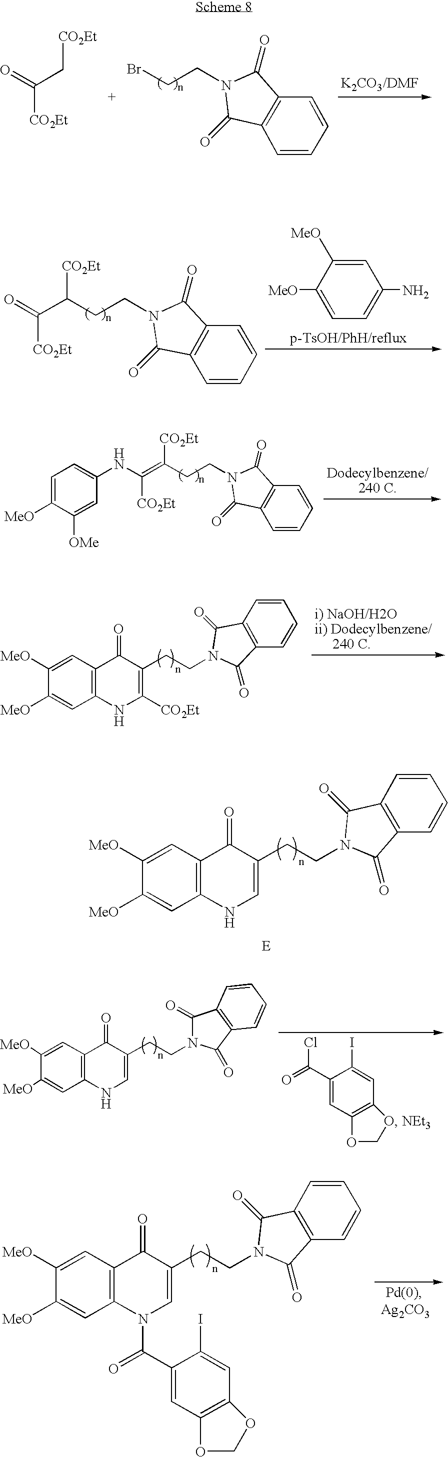

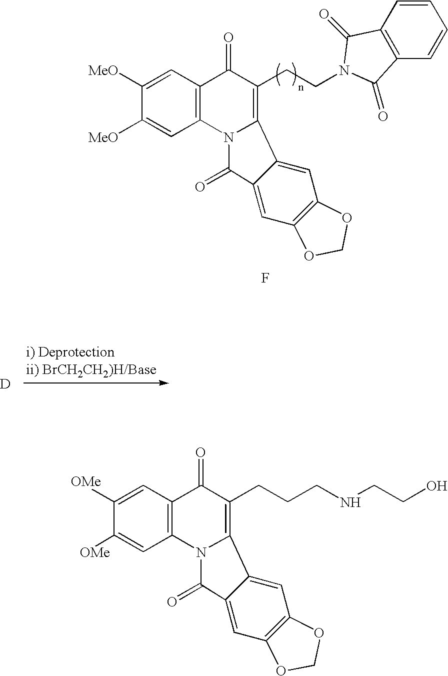

The phthalimide protecting group employed on the amine functionality in this synthesis can be replaced by other suitable protecting groups. Protecting groups for amines and other groups are described in Greene et al., Protective Groups in Organic Synthesis, John Wiley & Sons Inc., 3rd Edition, June 1999 (incorporated herein by reference). Also, the

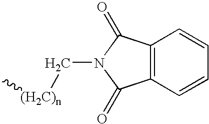

moiety in Scheme 8 can be replaced with a terminal alkenyl moiety

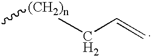

The terminal alkenyl moiety can be converted to an amino, alkylamino, or dialkylamino functionality following the method provided herein.

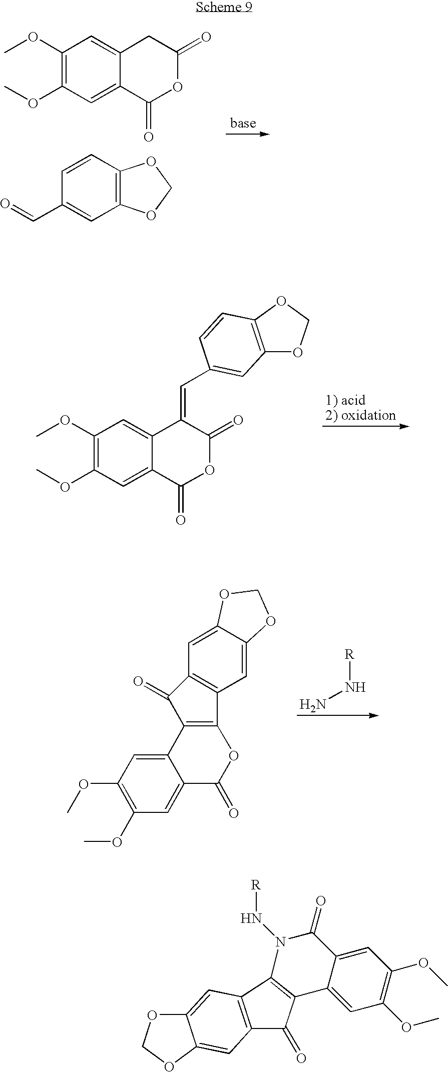

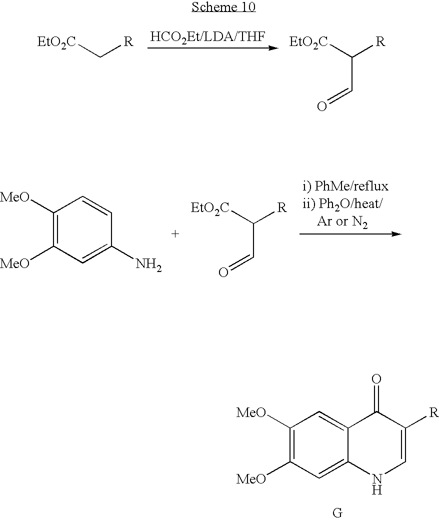

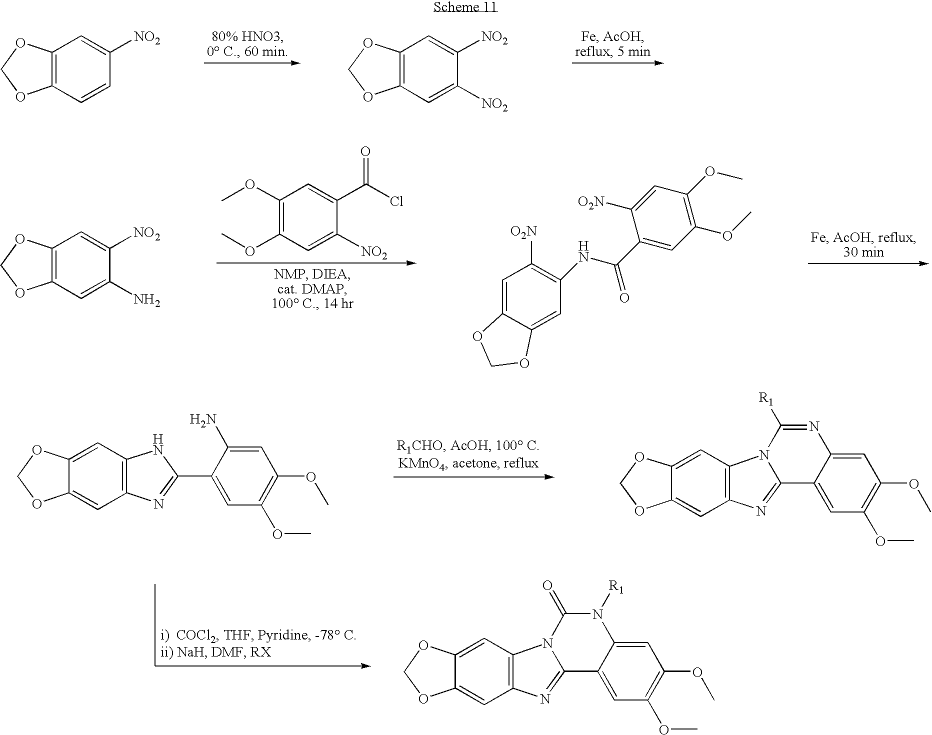

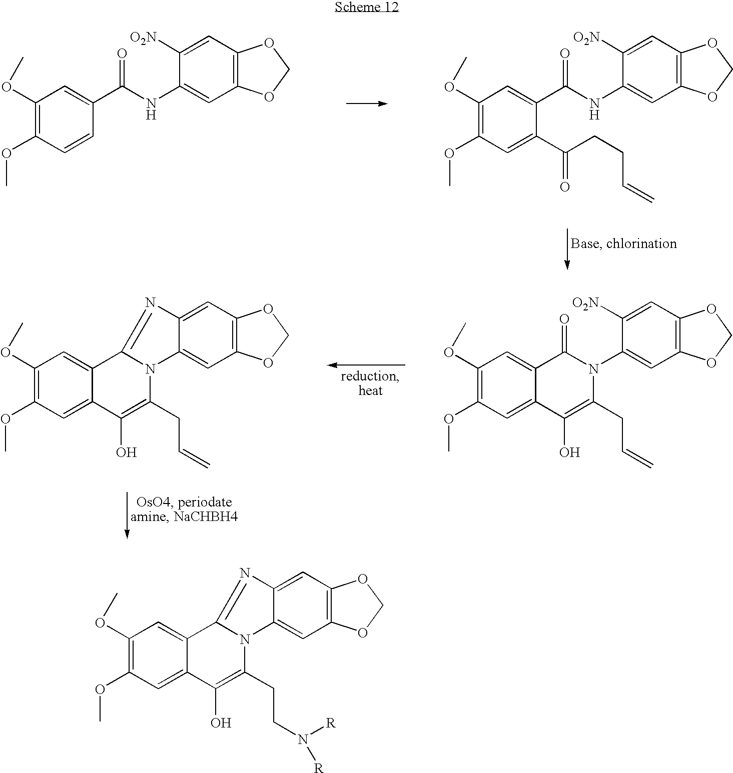

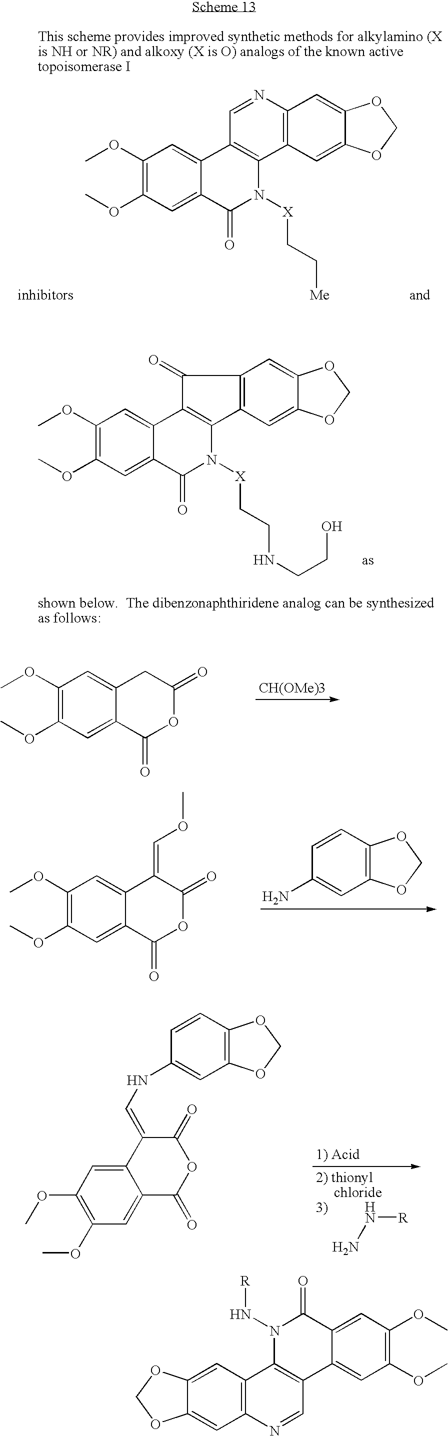

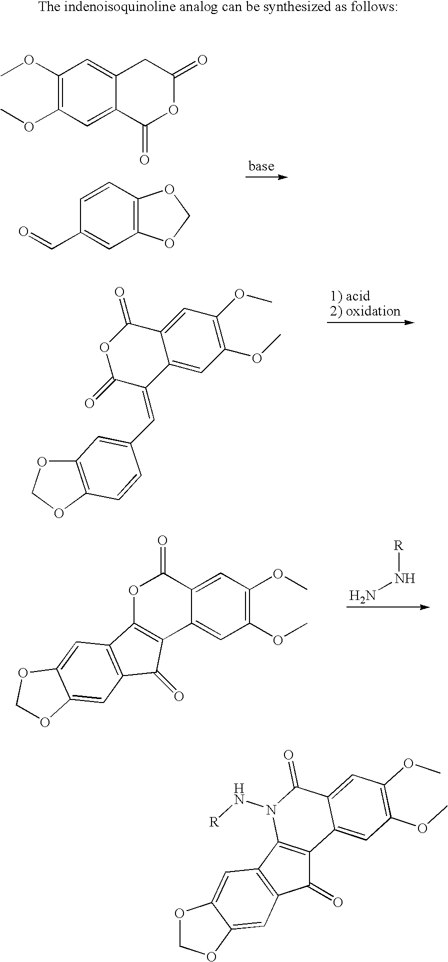

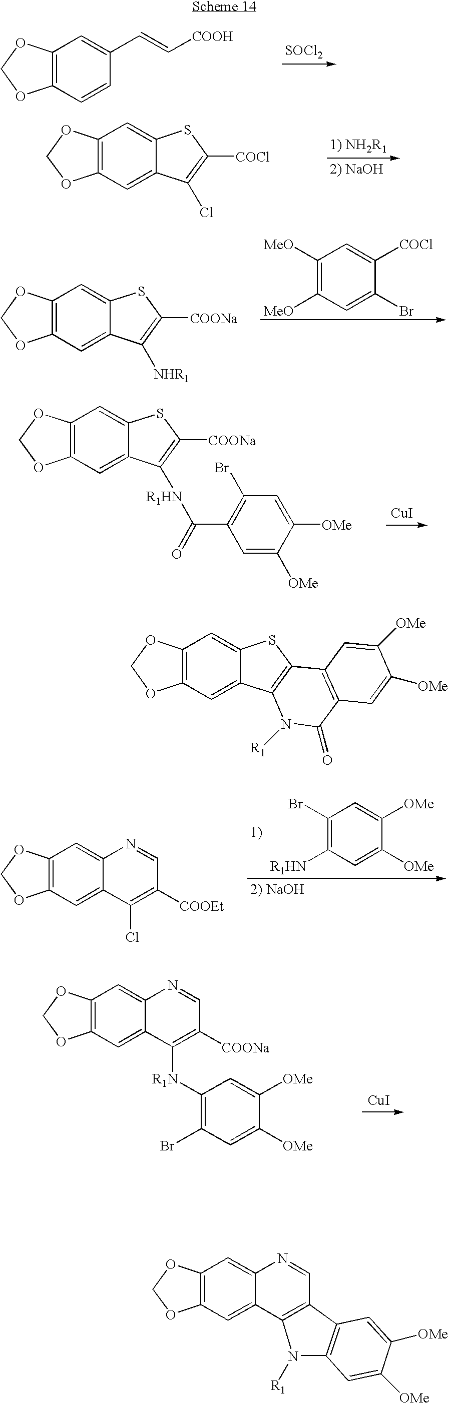

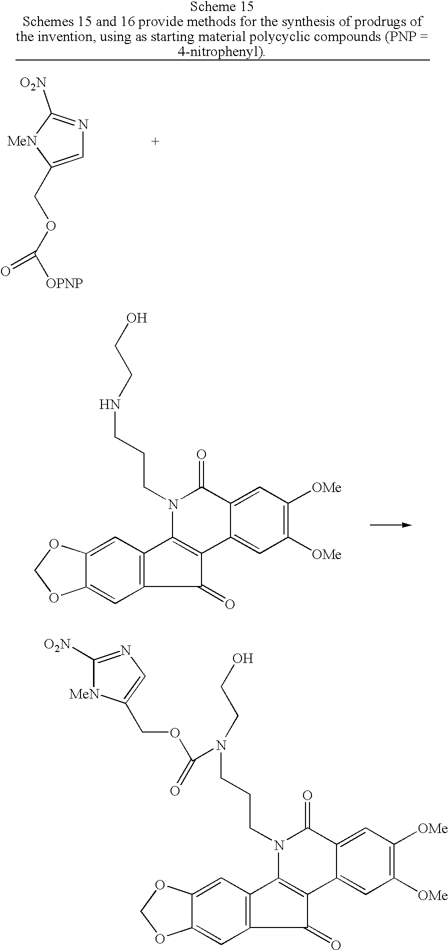

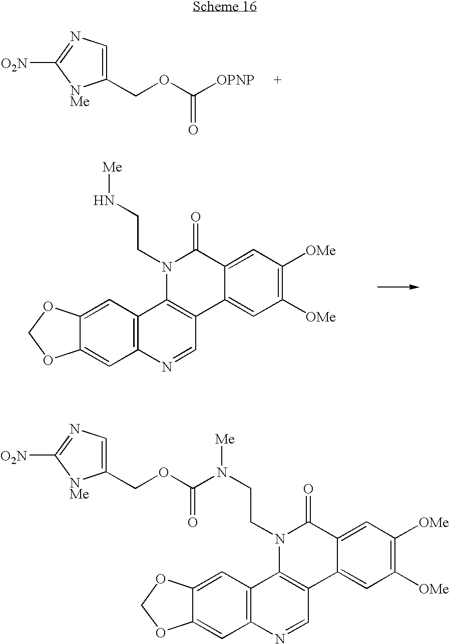

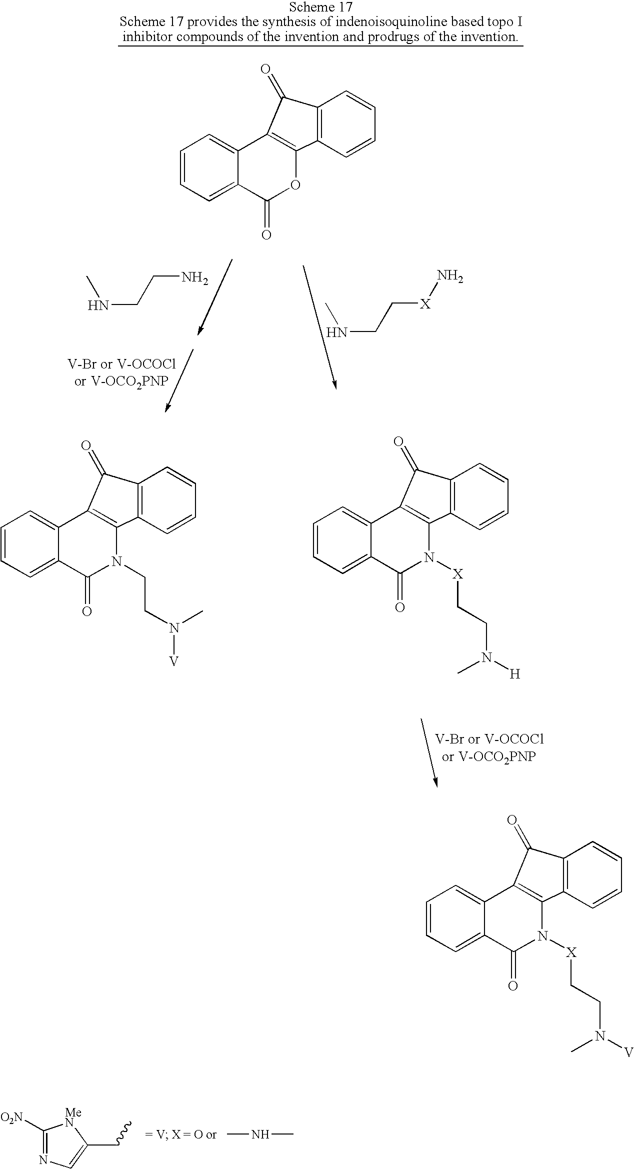

### Pharmaceutical Compositions

For use as a therapeutic agent, a compound of the present invention disclosed herein (including pharmaceutically acceptable salts, solvates, hydrates, and prodrugs) is usually formulated as a pharmaceutical composition comprising topoisomerase inhibitors and prodrugs of this invention) and a pharmaceutically-acceptable carrier. The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combining active compounds with solid excipient and, optionally, other compounds. Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. For topical or nasal administration, penetrants appropriate to the particular barrier to be permeated are used in the formulation. Such penetrants are generally known in the art.

Further details on techniques for formulation and administration can be found in the latest edition of Remington's Pharmaceutical Sciences (Maack Publishing Co., Easton, Pa.); GOODMAN AND GILMAN'S: THE PHARMACOLOGICAL BASIS OF THERAPEUTICS 10^{TH} EDITION 2001 by Louis Sanford Goodman et al., McGraw-Hill Professional; PHARMACEUTICAL DOSAGE FORMS AND DRUG DELIVERY SYSTEMS 7^{th} Edition Howard C. Ansel, et al., 2004, Lippincott Williams & Wilkins Publishers; PHARMACEUTICAL CALCULATIONS 11^{th} Edition, 2001, by Mitchell J. Stoklosa et al., Lippincott Williams & Wilkins; PHYSICAL PHARMACY: PHYSICAL CHEMICAL PRINCIPLES IN THE PHARMACEUTICAL SCIENCES 4^{th} Edition by Pilar Bustamante, et al., 1993, Lea & Febiger.

### Dosages and Administration

A variety of routes, dosage schedules, and dosage forms are appropriate for administration of pharmaceutical compositions of the invention. Appropriate dosage schedules and modes of administration will be apparent to the ordinarily skilled practitioner upon reading the present disclosure and/or can be determined using routine pharmacological methods and/or methods described herein.

The dose, schedule and duration of administration of the analog will depend on a variety of factors. The primary factor, of course, is the choice of a specific compound of the present invention. Other important factors include the age, weight and health of the subject, the severity of symptoms, if any, the subject's medical history, co-treatments, goal (e.g., prophylaxis or prevention of relapse), preferred mode of administration of the drug, the formulation used, patient response to the drug, and the like.

Additional guidance regarding the daily dose of administering a topoisomerase inhibitor and/or a prodrug of the invention can be obtained from similar administration information known for the topoisomerase inhibitor drug topotecan or a prodrug of a topoisomerase inhibitor, irinotecan. (See for example, GOODMAN AND GILMAN'S: THE PHARMACOLOGICAL BASIS OF THERAPEUTICS, supra). For example, a topoisomerase inhibitor and/or a prodrug of the invention can be administered, for treating cancer or other hyperproliferative diseases, at a dose in the range of about 0.25 mg to about 4000 mg of a compound of the invention per m² of body area of the patient to be treated per day, optionally with more than one dosage unit being administered per day, and typically with the daily dose being administered on multiple consecutive days. In one embodiment, the compounds of the present invention include novel compounds of the invention, novel prodrugs thereof, and novel prodrugs of known compounds. In one embodiment, a topoisomerase inhibitor of the invention is administered in a dose in the range of about 0.25 mg to about 100 mg per m² of body area of the patient to be treated per day. In another embodiment, a topoisomerase inhibitor is administered in a daily dose in the range of about 0.50 mg to about 50 mg per m² of body area of the patient to be treated. In certain other embodiments, a topoisomerase inhibitor is administered in a daily dose of about 0.75 to about 10 mg per m² of body area of the patient to be treated. In another embodiment, a daily dose is about 1 to about 5 mg per m² of body area of the patient to be treated.

In one embodiment, a prodrug of the invention is administered for treating cancer or other hyperproliferative diseases in a daily dose in the range of about 100 to about 3000 mg per m² of body area of the patient to be treated per day. In another embodiment, a prodrug of the invention is administered in a dose in the range of about 200 to about 2000 per m² of body area of the patient to be treated per day. In certain other embodiments, a prodrug of the invention is administered in a dose of about 500 to 1000 mg per m² of body area of the patient to be treated.

Additional guidance concerning administration is provided by prior experience using topoisomerase inhibitors and from new studies in humans and other mammals. Cell culture studies are frequently used in the art to optimize dosages, and the assays disclosed herein can be used in determining such doses.

For illustration, a therapeutically effective dose of a compound of the invention can be administered daily or once every other day or once a week to the patient. Controlled and sustained release formulations of the compound of the invention can be used. Generally, multiple administrations of the compound of the invention are employed. For optimum treatment benefit, the administration of the effective dose can be continued for multiple days, such as for at least five consecutive days, and often for at least a week and often for several weeks or more. In one embodiment, the compound of the invention is administered once (qday), twice (bid), three times (tid), or four times (qid) a day or once every other day (qod) or once a week (qweek), and treatment is continued for a period ranging from three days to two weeks or longer.

In one aspect, the present invention provides a method for treating cancer or other hyperproliferative diseases by administering to a patient in need of therapy thereof a therapeutically effective dose of a topoisomerase inhibitor of the invention to a patient in need of therapy thereof.

Of course modern cancer therapy often involves administering of a drug “cocktail” in which several anti-cancer drugs are contemporaneously administered to a cancer patient. The novel compounds of the present invention can be used in such therapies either in addition to or in substitution of one or more of the co-administered drugs. Also, because there may be cancer cells in a patient that are normoxic and located adjacent to a hypoxic region of a tumor, one can, in one embodiment of the invention, co-administering a prodrug of the invention with one or more other drugs that target normoxic cells.

In another embodiment, the hyperproliferative disease is selected from the group consisting of macular degeneration, gout, psoriasis, rheumatoid arthritis, restenosis, benign prostatic hyperplasia, and multiple sclerosis.

### Combination Therapies

In one embodiment, a compound and/or prodrug compound of the invention can be co-administered in combination with other anti-cancer agents (“anticancer agent”). Without intending to be bound by any particular mechanism or effect, such co-administration can in some cases provide one or more of several advantages over known cancer therapies, such as, for example co-administration of a compound and/or prodrug compound of the invention and the anticancer agent has a synergistic effect on induction of cancer cell death. Co-administration provides a better therapeutic result than administration of the anticancer agent alone, e.g., greater alleviation or amelioration of one or more symptoms of the cancer, diminishment of extent of disease, delay or slowing of disease progression, amelioration, palliation or stabilization of the disease state, partial or complete remission, prolonged survival or other beneficial therapeutic results.

The co-administration of a compound and/or a prodrug compound of the invention increases the sensitivity of cancer cells to the anticancer agent, allowing lower doses of the anticancer agent to be administered to the patient or allowing an anticancer agent to be used for treatment of cells otherwise resistant to the anticancer agent or otherwise refractory to treatment. Generally anti-cancer agents target rapidly dividing cells in the normoxic region, the prodrug compounds of the invention target the hypoxic cells in the regions of tumors that are not efficiently killed by the anticancer agent alone.

As used herein, a compound and/or a prodrug compound of the invention is “co-administered” with another anticancer agent (also referred to herein as, “Agent”) wherein a compound and/or a prodrug compound of the invention and Agent are administered as part of the same course of therapy. In one embodiment, a compound and/or a prodrug compound of the invention is first administered prior to administration of the Agent, (i.e., the initiation of the other cancer therapy), and treatment with the compound and/or prodrug compound of the invention is continued throughout the course of administration of the Agent (i.e., the course of the other therapy). In another embodiment, a compound and/or a prodrug compound of the invention is administered after the initiation or completion of the other cancer therapy. In other embodiments, a compound and/or a prodrug compound of the invention is first administered contemporaneously with the initiation of the other cancer therapy.

In one embodiment, a compound and/or a prodrug compound of the invention is first administered prior to administration of the Agent, and treatment with the compound and/or prodrug compound of the invention is continued after the cessation of administration of the Agent. In one embodiment, a compound and/or a prodrug compound of the invention is first administered prior to administration of the Agent, and treatment with the compound and/or prodrug compound of the invention is continued during part of the period of administration of the Agent. For certain drugs administration of a compound and/or a prodrug compound of the invention can be initiated and completed prior to the administration of the second drug.

In the presence of oxygen, the radical anion formed upon the reduction of hypoxia labile protecting group reacts with oxygen to yield superoxide and hypoxia labile protecting group. Superoxide is a cytotoxin and the production of superoxide in normoxic tissues can lead to unwanted side effects. In one embodiment, the present invention provides a method wherein a compound and/or a prodrug compound of the invention administered in combination with a chemoprotective agent or a chemoprotectant. Chemoprotective agents protect healthy tissue from the toxic effects of anticancer drugs. In one embodiment, the chemoprotective agent is a thiol or a disulfide. In one embodiment, the chemoprotectant can reduce superoxide. In another embodiment, the chemoprotectant can react with the “Michael-receptor” generated from a hypoxia activated prodrug of the invention and prevent “Michael-receptor” from reacting with proteins and nucleic acid.

Anticancer drug therapy today typically involves multiple rounds, or “cycles,” of administration of the anti-cancer agent(s). In the context of administering a compound and/or a prodrug compound of the invention, each cycle of administration (as well as a complete set of cycles) can be viewed as administration of a second drug. A compound and/or a prodrug compound of the invention can be administered in any or all of the multiple cycles of treatment with the other Agent; in general, the compound and/or prodrug compound of the invention is administered on a daily basis for at least two or more days during each cycle. In one aspect of the invention, a compound and/or a prodrug compound of the invention is co-administered with the Agent according to a schedule repeated at each round.

In one version of the method of treating cancer using the a compound and/or a prodrug compound of the invention, the compound and/or prodrug compound of the invention is administered in combination with an effective amount of one or more chemotherapeutic agents, an effective amount of radiotherapy, an appropriate surgery procedure, or any combination of such additional therapies.

When a compound and/or a prodrug compound of the invention is used in combination with one or more of the additional therapies, the compound and/or prodrug compound of the invention and additional therapy can be administered at the same time or can be administered separately. For example, if a compound and/or a prodrug compound of the invention is administered with an additional chemotherapeutic agent, the two agents can be administered simultaneously or can be administered sequentially with some time between administrations. One of skill in the art will understand methods of administering the agents simultaneously and sequentially and possible time periods between administrations.

The Agents can be administered as the same or different formulations and can be administered via the same or different routes.

Chemotherapeutic agents that can be used in combination with the compound of the invention include, but are not limited to, busulfan, improsulfan, piposulfan, benzodepa, carboquone, 2-deoxy-D-glucose, lonidamine and analogs thereof (reference apps), glufosfamide, meturedepa, uredepa, altretamine, imatinib, triethylenemelamine, triethylenephosphoramide, triethylenethiophosphoramide, trimethylolomelamine, chlorambucil, chlornaphazine, estramustine, ifosfamide, gefitinib, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elformithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, erlotonib, teniposide, tenuazonic acid, triaziquone, 2,2′,2″-trichlorotriethylamine, urethan, vinblastine, cyclophosphamide, and vincristine. Other chemotherapeutic agents that can be used include platinum derivatives, including but not limited to cis platinum, carboplatin, and oxoplatin.

In one version, a compound and/or a prodrug compound of the invention can be used in combination with an angiogenesis inhibitor including but not limited to Avastin and similar therapeutics. In one version of the combination treatment methods, a subject is treated with an angiogenisis inhibitor and subsequently treated with a compound and/or a prodrug compound of the invention. In one version of these combination methods of treatment using an angiogenesis inhibitor, the method is used to treat breast cancer.

In another embodiment, a compound and/or a prodrug compound of the invention is administered with an anti-cancer agent that acts, either directly or indirectly, to inhibit the epidermal growth factor or EGFR receptor. EGFR inhibitors suitable for coadministration with a compound of the invention include gefitinib and erlotonib.

In another version, a compound and/or a prodrug compound of the invention is administered with an anti-cancer agent that acts, either directly or indirectly, to inhibit hypoxia-inducible factor 1 alpha (HIF1a) or to inhibit a protein or enzyme, such as a glucose transporter or VEGF, whose expression or activity is increased upon increased HIF1a levels. HIF1a inhibitors suitable for use in this version of the methods and compositions described herein include P13 kinase inhibitors; LY294002; rapamycin; histone deacetylase inhibitors such as [(E)-(1S,4S,10S,21R)-7-[(Z)-ethylidene]-4,21-diisopropyl-2-oxa-12,13-dithia-5,8,20,23-tetraazabicyclo-[8,7,6]-tricos-16-ene-3,6,9,19,22-pentanone (FR901228, depsipeptide); heat shock protein 90 (Hsp90) inhibitors such as geldanamycin, 17-allylamino-geldanamycin (17-MG), and other geldanamycin analogs, and radicicol and radicicol derivatives such as KF58333; genistein; indanone; staurosporin; protein kinase-1 (MEK-1) inhibitors such as PD98059 (2′-amino-3′-methoxyflavone); PX-12 (1-methylpropyl 2-imidazolyl disulfide); pleurotin PX-478; quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins; barbituric and thiobarbituric acid analogs; camptothecins; and YC-1, a compound described in Biochem. Pharmacol., 15 Apr. 2001, 61(8):947-954, incorporated herein by reference, and its derivatives.

In another version, a compound and/or a prodrug compound of the invention is administered with an anti-angiogenic agent, including but not limited to anti-angiogenic agents selected from the group consisting of angiostatin, an agent that inhibits or otherwise antagonizes the action of VEGF, batimastat, captopril, cartilage derived inhibitor, genistein, endostatin, interleukin, lavendustin A, medroxypregesterone acetate, recombinant human platelet factor 4, Taxol, tecogalan, thalidomide, thrombospondin, TNP-470, and Avastin. Other useful angiogenesis inhibitors for purposes of the combination therapies provided by the present methods and compositions described herein include Cox-2 inhibitors like celecoxib (Celebrex), diclofenac (Voltaren), etodolac (Lodine), fenoprofen (Nalfon), indomethacin (Indocin), ketoprofen (Orudis, Oruvail), ketoralac (Toradol), oxaprozin (Daypro), nabumetone (Relafen), sulindac (Clinoril), tolmetin (Tolectin), rofecoxib (Vioxx), ibuprofen (Advil), naproxen (Aleve, Naprosyn), aspirin, and acetaminophen (Tylenol).

In addition, because pyruvic acid plays an important role in angiogenesis, pyruvate mimics and glycolytic inhibitors like halopyruvates, including bromopyruvate, can be used in combination with an anti-angiogenic compound and a compound and/or a prodrug compound of the invention to treat cancer. In another version, a compound and/or a prodrug compound of the invention is administered with an anti-angiogenic agent and another anti-cancer agent, including but not limited to a cytotoxic agent selected from the group consisting of alkylators, Cisplatin, Carboplatin, and inhibitors of microtubule assembly, to treat cancer.

In addition to the combination of a compound and/or a prodrug compound of the invention with the Agents described above, the present methods and compositions described herein provides a variety of synergistic combinations of a compound and/or a prodrug compound of the invention and other anti-cancer drugs. Those of skill in the art can readily determine the anti-cancer drugs that act “synergistically” with a compound and/or a prodrug compound of the invention as described herein. For example, the reference Vendetti, “Relevance of Transplantable Animal-Tumor Systems to the Selection of New Agents for Clinical Trial,” Pharmacological Basis of Cancer Chemotherapy, Williams and Wilkins, Baltimore, 1975, and Simpson Herren et al., 1985, “Evaluation of In Vivo Tumor Models for Predicting Clinical Activity for Anticancer Drugs,” Proc. Am. Assoc. Cancer Res. 26: 330, each of which is incorporated herein by reference, describe methods to aid in the determination of whether two drugs act synergistically.

While synergy is not required for therapeutic benefit in accordance with the methods of described herein, in one embodiment, the present invention provides a method of cancer treatment, wherein there is synergy between a compound and/or a prodrug compound of the invention and another anticancer agent. Two drugs can be said to possess therapeutic synergy if a combination dose regimen of the two drugs produces a significantly better tumor cell kill than the sum of the single Agents at optimal or maximum tolerated doses. The “degree of synergy” can be defined as net log of tumor cell kill by the optimum combination regimen minus net log of tumor cell kill by the optimal dose of the most active single Agent. Differences in cell kill of greater than ten-fold (one log) are considered conclusively indicative of therapeutic synergy.

When a compound and/or a prodrug compound of the invention is used with another anti-cancer agent, the compound and/or prodrug compound of the invention will, at least in some versions, be administered prior to the initiation of therapy with the other drug or drugs and administration will typically be continued throughout the course of treatment with the other drug or drugs. In some versions, the drug co-administered with a compound and/or a prodrug compound of the invention will be delivered at a lower dose, and optionally for longer periods, than would be the case in the absence of administering the compound and/or prodrug of the invention. Such “low dose” therapies can involve, for example, administering an anti-cancer drug, including but not limited to paclitaxel, docetaxel, doxorubicin, cisplatin, or carboplatin, at a lower than approved dose and for a longer period of time together with a compound and/or a prodrug compound of the invention administered in accordance with the methods described herein.

These methods can be used to improve patient outcomes over currently practiced therapies by more effectively killing cancer cells or stopping cancer cell growth as well as diminishing unwanted side effects of the other therapy. In other versions, the other anti-cancer agent or agents will be administered at the same dose levels used when a compound and/or a prodrug compound of the invention is not co-administered. When employed in combination with a compound and/or a prodrug compound of the invention, the additional anti-cancer agent(s) is dosed using either the standard dosages employed for those Agents when used without the compound and/or prodrug compound of the invention or are less than those standard dosages.

The administration of a compound and/or a prodrug compound of the invention in accordance with the methods described herein can therefore allow the physician to treat cancer with existing (or later approved) drugs at lower doses (than currently used), thus ameliorating some or all of the toxic side effects of such drugs. The exact dosage for a given patient varies from patient to patient, depending on a number of factors including the drug combination employed, the particular disease being treated, and the condition and prior history of the patient, but can be determined using only the skill of the ordinarily skilled artisan in view of the teachings herein.

Specific dose regimens for known and approved chemotherapeutic agents or antineoplastic agents (i.e., the recommended effective dose) are known to physicians and are given, for example, in the product descriptions found in the Physician's Desk Reference 2003, (Physicians' Desk Reference, 57th Ed) Medical Economics Company, Inc., Oradell, N.J. and/or are available from the Federal Drug Administration. Illustrative dosage regimens for certain anti-cancer drugs are also provided below.

Cancer drugs can be classified generally as alkylators, anthracyclines, antibiotics, aromatase inhibitors, bisphosphonates, cyclo-oxygenase inhibitors, estrogen receptor modulators, folate antagonists, inorganic aresenates, microtubule inhibitors, modifiers, nitrosoureas, nucleoside analogs, osteoclast inhibitors, platinum containing compounds, retinoids, topoisomerase 1 inhibitors, topoisomerase 2 inhibitors, and tyrosine kinase inhibitors. In accordance with the methods described herein, a compound and/or a prodrug compound of the invention can be co-administered with any anti-cancer drug from any of these classes or can be administered prior to or after treatment with any such drug or combination of such drugs. In addition, a compound and/or a prodrug compound of the invention can be administered in combination with a biologic therapy (e.g., treatment with interferons, interleukins, colony stimulating factors and monoclonal antibodies). Biologics used for treatment of cancer are known in the art and include, for example, trastuzumab (Herceptin), tositumomab and ¹³¹I Tositumomab (Bexxar), rituximab (Rituxan).

Alkylators useful in the practice of the methods described herein include but are not limited to busulfan (Myleran, Busulfex), chlorambucil (Leukeran), ifosfamide (with or without MESNA), cyclophosphamide (Cytoxan, Neosar), glufosfamide, melphalan, L-PAM (Alkeran), dacarbazine (DTIC-Dome), and temozolamide (Temodar). In accordance with the methods described herein a compound and/or a prodrug compound of the invention is co-administered with an alkylator to treat cancer. In one version, the cancer is chronic myelogenous leukemia, multiple myeloma, or anaplastic astrocytoma.

In one embodiment, the present invention provides a method of treating cancer treatable by administering a compound and/or a prodrug compound of the invention of the present invention alone or in combination with at least another alkylator or a prodrug thereof. Alkylators, such as, for example, cyclophosphamide, ifosfamide, glufosfamide, mechlorethamine, melphalan, chlorambucil, dacarbazine, temozolomide, carmustine, streptozocin, bendamustin, busulfan, thiotepa, cisplatin, carboplatin, and oxaliplatin, and types of cancers treated using any one of such alkylators alone or in combination with other anti cancer or chemoprotective agents are described for example in the reference Hardman et al., (see Hardman et al., The Pharmacological Basis of Therapeutics, 2001, 1389-1399, McGraw-Hill, New York, USA).

In one embodiment, the present invention provides a method of treating cancer by administering a compound and/or a prodrug compound of the invention with a cancer treatment regimen using at least the alkylator Glufosfamide. Glufosfamide is in the clinic for the treatment of pancreatic cancer or Gemzar resistant pancreatic cancer. Glufosfamide can be used for treating breast cancer, Morbus Hodgkin, gastrointestinal tract cancer, or as part of the GCE (Glufosfamide, Carboplatin, and Etoposide) or RGCE (Rituxan and GCE) regimen, for treating lymphomas. (Tidmarsh et al., U.S. Pat. Appl. No. 60/638,995, 60/680,451 and 60/719,787). Additional examples of Agents include Terciva, Iressa, Cytarabine and Erbitux.

In one embodiment, the present invention provides a method of treating cancer by administering a compound and/or a prodrug compound of the invention with a cancer treatment regimen using at least a platinum coordination complex alkylator. In one embodiment, the platinum coordination complex alkylator is Cisplatin. Cisplatin can be used to treat cancer of bladder, head and neck, endometrium, small cell carcinoma of the lung, and some neoplasms of childhood. Cisplatin alone or with cyclophosphamide is used to treat advanced ovarian cancer. Combination chemotherapy of Cisplatin with Bleomycin, Etoposide, and Vinblastine is used to treat advanced testicular cancer; and with one of Paclitaxel, Cyclophosphamide, or Doxorubicin to treat ovarian carcinoma.

Anthracyclines useful in the practice of the methods described herein include but are not limited to, doxorubicin (Adriamycin, Doxil, Rubex), mitoxantrone (Novantrone), idarubicin (Idamycin), valrubicin (Valstar), and epirubicin (Ellence). In accordance with the methods described herein a compound and/or a prodrug compound of the invention is co-administered with an anthracycline to treat cancer. In one version, the cancer is acute nonlymphocytic leukemia, Kaposi's sarcoma, prostate cancer, bladder cancer, metastatic carcinoma of the ovary, and breast cancer.

As one example the compound (8S,10S)-10-[(3-Amino-2,3,6-trideoxy-alpha.-L-lyxo-hexopyranosyl)oxy]-8-glycoloyl-7,8,9,10-tetrahydro-6,8,11-trihydroxy-1-methoxy-5,12-naphthacenedione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumor, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma. Doxorubicin is typically administered in a dose in the range of 30-75 mg/m² as a single intravenous injection administered at 21-day intervals; weekly intravenous injection at doses of 20 mg/m²; or 30 mg/m² doses on each of three successive days repeated every four weeks. In accordance with the methods of the methods described herein, a compound and/or a prodrug compound of the invention is co-administered starting prior to and continuing after the administration of doxorubicin at such doses (or at lower doses). Cyclic Anthracycline cytotoxin prodrugs useful in the practice of the methods described herein are provided by the reference Matteuci et al., PCT Patent Application No. US05/08161.

Antibiotics useful in the practice of the methods described herein include but are not limited to dactinomycin, actinomycin D (Cosmegen), bleomycin (Blenoxane), daunorubicin, and daunomycin (Cerubidine, DanuoXome). In accordance with the methods described herein a compound and/or a prodrug compound of the invention is co-administered with an antibiotic to treat cancer. In one version, the cancer is a cancer selected from the group consisting of acute lymphocytic leukemia, other leukemias, and Kaposi's sarcoma.

Aromatase inhibitors useful in the practice of the methods described herein include but are not limited to anastrozole (Arimidex) and letroazole (Femara). In accordance with the methods described herein a compound and/or a prodrug compound of the invention is co-administered with an aromatase inhibitor to treat cancer. In one version, the cancer is breast cancer.

Bisphosphonate inhibitors useful in the practice of the methods described herein include but are not limited to zoledronate (Zometa). In accordance with the methods described herein a compound and/or a prodrug compound of the invention is co-administered with a biphosphonate inhibitor to treat cancer. In one version, the cancer is a cancer selected from the group consisting of multiple myeloma, bone metastases from solid tumors, or prostate cancer.

Cyclo-oxygenase inhibitors useful in the practice of the methods described herein include but are not limited to celecoxib (Celebrex). In accordance with the methods described herein a compound and/or a prodrug compound of the invention is co-administered with a cyclo-oxygenase inhibitor to treat cancer. In one version, the cancer is colon cancer or a pre-cancerous condition known as familial adenomatous polyposis.

Estrogen receptor modulators useful in the practice of the methods described herein include but are not limited to tamoxifen (Nolvadex) and fulvestrant (Faslodex). In accordance with the methods described herein a compound and/or a prodrug compound of the invention is co-administered with an estrogen receptor modulator to treat cancer. In one version, the cancer is breast cancer or the treatment is administered to prevent the occurrence or reoccurrence of breast cancer.

Folate antagonists useful in the practice of the methods described herein include but are not limited to methotrexate and tremetrexate. In accordance with the methods described herein a compound and/or a prodrug compound of the invention is co-administered with a folate antagonist to treat cancer. In one version, the cancer is osteosarcoma.

As one example, the compound N-[4-[[(2,4-diamino-6-pteridinyl)methyl methylamino]benzoyl]-L-glutamic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides. Methotrexate is administered as follows. For choriocarcinoma, intramuscular injections of doses of 15 to 30 mg are administered daily for a five-day course, such courses repeated as needed with rest period of one or more weeks interposed between courses of therapy. For leukemias, twice weekly intramuscular injections are administered in doses of 30 mg/m². For mycosis fungoides, weekly intramuscular injections of doses of 50 mg or, alternatively, of 25 mg are administered twice weekly. In accordance with the methods described herein, a compound and/or a prodrug compound of the invention is co-administered with methotrexate administered at such doses (or at lower doses). 5-Methyl-6-[[(3,4,5-trimethoxyphenyl)-amino]methyl]-2,4-quinazolinediamine (commonly known as trimetrexate) is another antifolate drug that can be co-administered with a compound and/or a prodrug compound of the invention.

Inorganic arsenates useful in the practice of the methods described herein include but are not limited to arsenic trioxide (Trisenox). In accordance with the methods described herein a compound and/or a prodrug compound of the invention is co-administered with an inorganic arsenate to treat cancer. In one version, the cancer is refractory acute promyelocytic leukemia (APL).

Microtubule inhibitors (as used herein, a “microtubule inhibitor” is any agent that interferes with the assembly or disassembly of microtubules) useful in the practice of the methods described herein include but are not limited to vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. Tubulin binding anticancer drugs and prodrugs thereof which can be used in the practice of the methods of the present invention are provided in the reference Matteucci et al., U.S. Patent Application No. 60/630,422. In accordance with the methods described herein a compound of the invention is co-administered with a microtubule inhibitor to treat cancer. In one version, the cancer is ovarian cancer, breast cancer, non-small cell lung cancer, Kaposi's sarcoma, and metastatic cancer of breast or ovary origin. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease, lymphosarcoma, reticulum-cell sarcoma, rhabdomyosarcoma, neuroblastoma, and Wilm's tumor. Vincristine is administered in weekly intravenous doses of 2 mg/m² for children and 1.4 mg/m² for adults. In accordance with the methods described herein, a compound and/or prodrug compound of the invention is co-administered with vincristine administered at such doses. In one version, a compound and/or prodrug compound of the invention is not administered prior to treatment with a microtubule inhibitor, such as a taxane, but rather, administration of a compound and/or prodrug compound of the invention is administered simultaneously with or within a few days to a week after initiation of treatment with a microtubule inhibitor.

Modifiers useful in the practice of the methods described herein include but are not limited to Leucovorin (Wellcovorin), which is used with other drugs such as 5-fluorouracil to treat colorectal cancer. In accordance with the methods described herein a compound and/or prodrug compound of the invention is co-administered with a modifier and another anti-cancer agent to treat cancer. In one version, the cancer is colon cancer. In one version, the modifier is a compound that increases the ability of a cell to take up glucose, including but not limited to the compound N-hydroxyurea. N-hydroxyurea has been reported to enhance the ability of a cell to take up 2-deoxyglucose (see the reference Smith et al., 1999, Cancer Letters 141: 85, incorporated herein by reference), and administration of N-hydroxyurea at levels reported to increase 2-deoxyglucose uptake or to treat leukemia together with administration of 2-deoxyglucose and a compound of the invention is one version of the therapeutic methods provided herein. In another such version, a compound and/or prodrug compound of the invention is co-administered with nitric oxide or a nitric oxide precursor, such as an organic nitrite or a spermineNONOate, to treat cancer, as the latter compounds stimulate the uptake of glucose.

Nitrosoureas useful in the practice of the methods described herein include but are not limited to procarbazine (Matulane), lomustine, CCNU (CeeBU), carmustine (BCNU, BiCNU, Gliadel Wafer), and estramustine (Emcyt). In accordance with the methods described herein a compound and/or prodrug compound of the invention is co-administered with a nitrosourea to treat cancer. In one version, the cancer is prostate cancer or glioblastoma, including recurrent glioblastoma multiforme.

Nucleoside analogs useful in the practice of the methods described herein include but are not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), azacytidine (Vidaza), pentostatin (Nipent), cladribine (Leustatin, 2-CdA), gemcitabine (Gemzar), and capecitabine (Xeloda). In accordance with the methods described herein a compound and/or prodrug compound of the invention is co-administered with a nucleoside analog to treat cancer. In one version, the cancer is B-cell lymphocytic leukemia (CLL), hairy cell leukemia, adenocarcinoma of the pancreas, metastatic breast cancer, non-small cell lung cancer, or metastatic colorectal carcinoma. As one example, the compound 5-fluoro-2,4(1H,3H)-pyrimidinedione, also commonly known as 5-fluorouracil, is an antimetabolite nucleoside analog effective in the palliative management of carcinoma of the colon, rectum, breast, stomach, and pancreas in patients who are considered incurable by surgical or other means. 5-Fluorouracil is administered in initial therapy in doses of 12 mg/m² given intravenously once daily for 4 successive days with the daily dose not exceeding 800 mg. If no toxicity is observed at any time during the course of the therapy, 6 mg/kg are given intravenously on the 6th, 8th, 10th, and 12th days. No therapy is given on the 5th, 7th, 9th, or 11th days. In poor risk patients or those who are not in an adequate nutritional state, a daily dose of 6 mg/kg is administered for three days, with the daily dose not exceeding 400 mg. If no toxicity is observed at any time during the treatment, 3 mg/kg can be given on the 5th, 7th, and 9th days. No therapy is given on the 4th, 6th, or 8th days. A sequence of injections on either schedule constitutes a course of therapy. In accordance with the methods described herein, a compound and/or prodrug compound of the invention is co-administered with 5-FU administered at such doses or with the prodrug form Xeloda with correspondingly adjusted doses. As another example, the compound 2-amino-1,7-dihydro-6H-purine-6-thione, also commonly known as 6-thioguanine, is a nucleoside analog effective in the therapy of acute non-pymphocytic leukemias. 6-Thioguanine is orally administered in doses of about 2 mg/kg of body weight per day. The total daily dose can be given at one time. If after four weeks of dosage at this level there is no improvement, the dosage can be cautiously increased to 3 mg/kg/day. In accordance with the methods described herein, a compound and/or prodrug compound of the invention is co-administered with 6-TG administered at such doses (or at lower doses).

Osteoclast inhibitors useful in the practice of the methods described herein include but are not limited to pamidronate (Aredia). In accordance with the methods described herein a compound and/or prodrug compound of the invention is co-administered with an osteoclast inhibitor to treat cancer. In one version, the cancer is osteolytic bone metastases of breast cancer, and one or more additional anti-cancer agents are also co-administered with a compound and/or prodrug compound of the invention.

Platinum compounds useful in the practice of the methods described herein include but are not limited to cisplatin (Platinol) and carboplatin (Paraplatin). In accordance with the methods described herein a compound and/or prodrug compound of the invention is co-administered with a platinum compound to treat cancer. In one version, the cancer is metastatic testicular cancer, metastatic ovarian cancer, ovarian carcinoma, and transitional cell bladder cancer. As one example, the compound cis-Diaminedichloroplatinum (II), commonly known as cisplatin, is useful in the palliative treatment of metastatic testicular and ovarian tumors, and for the treatment of transitional cell bladder cancer which is not amenable to surgery or radiotherapy. Cisplatin, when used for advanced bladder cancer, is administered in intravenous injections of doses of 50-70 mg/m² once every three to four weeks. In accordance with the methods described herein, a compound and/or prodrug compound of the invention is co-administered with cisplatin administered at these doses (or at lower doses). One or more additional anti-cancer agents can be co-administered with the platinum compound and a compound and/or prodrug compound of the invention. As one example, Platinol, Blenoxane, and Velbam can be co-administered with a compound and/or prodrug compound of the invention. As another example, Platinol and Adriamycin can be co-administered with a compound and/or prodrug compound of the invention.

Retinoids useful in the practice of the methods described herein include but are not limited to tretinoin, ATRA (Vesanoid), alitretinoin (Panretin), and bexarotene (Targretin). In accordance with the methods described herein a compound and/or prodrug compound of the invention is co-administered with a retinoid to treat cancer. In one version, the cancer is a cancer selected from the group consisting of APL, Kaposi's sarcoma, and T-cell lymphoma.

In accordance with the methods described herein a compound and/or prodrug compound of the invention is co-administered with other topoisomerase inhibitors to treat cancer. Examples of topoisomerase I inhibitors useful in the practice of the methods described herein include but are not limited to topotecan (Hycamtin) and irinotecan (Camptostar). Examples of topoisomerase 2 inhibitors useful in the practice of the methods described herein include but are not limited to etoposide, VP-16 (Vepesid), teniposide, VM-26 (Vumon), and etoposide phosphate (Etopophos). In one version, the cancer is metastatic carcinoma of the ovary, colon, or rectum, or small cell lung cancer. In one version, the cancer is a cancer selected from the group consisting of refractory testicular tumors, refractory acute lymphoblastic leukemia (ALL), and small cell lung cancer. As noted above, however, in one version of the methods described herein, administration of a compound and/or prodrug compound of the invention either precedes or follows, or both, administration of a topoisomerase inhibitor but is not administered concurrently therewith.

Tyrosine kinase inhibitors useful in the practice of the methods described herein include but are not limited to imatinib (Gleevec). In accordance with the methods described herein a compound and/or prodrug compound of the invention is co-administered with a tyrosine kinase inhibitor to treat cancer. In one version, the cancer is CML or a metastatic or unresectable malignant gastrointestinal stromal tumor.

Lonidamine analogs useful in the practice of the present invention are provides in the reference PCT Pat. Appl. Nos. PCT/US2005/026929 and PCT/US2005/027092 and PCT/US2005/024434.

Thus, described herein are methods of treating cancer in which a compound and/or prodrug compound of the invention or a pharmaceutically acceptable salt thereof and one or more additional anti-cancer agents are administered to a patient. Specific versions of such other anti-cancer agents include without limitation 5-methyl-6-[[(3,4,5-trimethoxyphenyl)amino]-methyl]-2,4-quinazolinediamine or a pharmaceutically acceptable salt thereof, (8S,10S)-O-(3-amino-2,3,6-trideoxy-alpha-L-lyxo-hexopyranosyl)oxy]-8-glycoloyl-7,8,9,10-tetrahydro-6,8,11-trihydroxy-1-methoxy-5,12-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]-methylamino]benzoyl]-L-glutamic acid, or a pharmaceutically acceptable salt thereof; or cisdiamminedichloro-platinum (II).

Functional characteristics of topoisomerase inhibitors. The topoisomerase inhibitors and prodrugs suited for use in the invention are those that interfere with the topoismerase enzyme when administered to a human, non-human primate, or other mammal. As is usual in the pharmaceutical arts, not every structural analog of a compound (e.g., a topo I inhibitor) is pharmacologically active. Active forms can be identified by routine screening of analogs for the activity of the parent compound. A variety of assays and tests can be used to assess pharmacological activity of a topo I inhibitor, including in vitro assays, such as those described below and elsewhere herein, in vivo assays in humans, non-human primates and other mammals, and/or clinical studies.

In some embodiments of the invention in which topoisomerase inhibitor is used for treatment or prevention of cancer or its manifestations, a topoisomerase inhibitor with similar apoptosis-inducing activity similar to that of topotecan is selected. Thus, in some embodiments of the invention, a topoisomerase inhibitor that induces apoptosis in cancer cells such as H460, PC3, CCRF, LNCaP, HT29, MESSA and PWR-1E is administered to treat cancer.

In some embodiments of the invention in which topoisomerase inhibitor is used for treatment or prevention of a hyperproliferative disease or its manifestations, topoisomerase inhibitor with similar apoptosis-inducing activity similar to that of topotecan is selected. Thus, in some embodiments of the invention, a topoisomerase inhibitor that induces apoptosis in skin, epithelial or endothelial, nerve, and T cells, is administered to treat a hyperproliferative disease, e.g. psoriasis, rheumatoid arthritis, restenosis, benign prostatic hyperplasia, and multiple sclerosis.

HIF-1α expression assays. The hypoxia-inducible factor-1 (HIF-1) transcription factor is an essential regulator of tumorigenesis. Topoisomerase inhibitors reduced HIF-1α expression/accumulation (measured in the nuclear fraction) in multiple human cancer cell lines (H460, PC3, CCRF), wherein the cells are cultured under conditions of hypoxia. Thus, in some embodiments of the invention, a topoisomerase inhibitor reduces HIF-1α expression (prevents HIF-1α accumulation) in H460, PC3, CCRF cells cultured under hypoxic conditions.

Clonogenic Assays for determining cytotoxicity of the compounds of the invention: Cytotoxicity of the compounds of the invention can be determined in hypoxia and in normoxia by clonogenic assay employing, for example, H460 and HT29 cell lines and can be expressed as IC₉₀ in μM, and by anti-proleferation assay performed by modifying a multi-well assay described by Hay et al., J. Med. Chem., 2003, 46:169-82 employing H460, HT29, HCT116, and DX-5 cell lines and can be expressed as IC₅₀ in μM. The ratio of IC₅₀ or IC₉₀ determined in normoxia and hypoxia is called hypoxia cytotoxicity ratio (HCR) and can be a measure of the hypoxia selective cytotoxicity of the prodrugs of the present invention.

### EXAMPLES

### Example 1

### Synthesis of Compounds 7 and 8

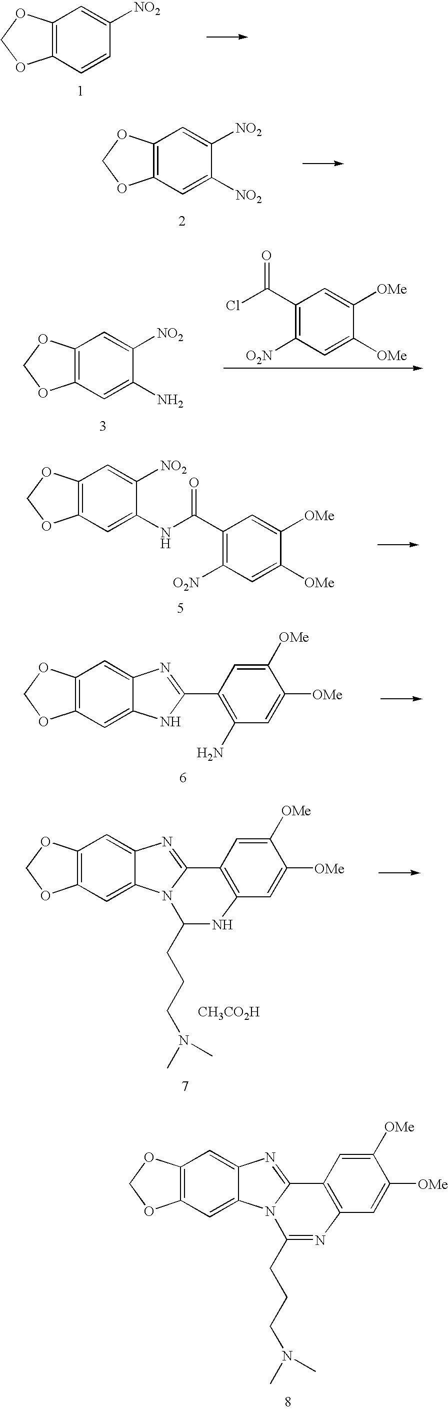

Fuming nitric acid (80%, 16.7 mL) was added dropwise into compound 1 contained in a round bottom flask (25 mL) chilled in ice-NaCl dewar (−10° C.). Temperature was raised to −5° C. while stirring. After an hour, a small amount of reaction mixture was worked up by adding to ice water, extracted with dichloromethane (DCM), and analyzed by thin layer chromatography (ethyl acetate (EtOAc)/Hexane) to show completion of reaction. The reaction mixture was added in 1 mL portions to 100 mL ice water with stirring. The precipitate was filtered using a fine frit washed with water (3×60 mL). A small amount of solid NaHCO₃ was added to the residue, the residue washed with water (3×30 mL), and dried in vacuo to yield compound 2 (1.28 g, 88% yield).

A solution of compound 2 (4.29 g) in glacial acetic acid (57 mL) was heated to boiling, heating stopped, and iron powder (3.38 g) added to it. After a vigorous exothermic reaction subsided, the mixture was refluxed at 145° C. for 5 minutes, poured into ice/water (400 mL) while stirring and the precipitate filtered using a fine frit. The residue was washed with water (3×100 mL) and dried in vacuo overnight to yield compound 3 (2.96 g) whose structure was confirmed by NMR.

To a solution of compound 3 (1.08 g) and 4 (1.74 g), obtained by reacting compound 3 in THF with oxalyl chloride and a catalytic amount of DMF and removing solvent in vacuo) in N-methylpyrrolidone (NMP, 15 mL), diisopropylethylamine (2.5 mL) and N,N-dimethylaminopyridine (145 mg) was added and stirred for 4 h at 100° C. The reaction mixture was cooled to rt and diluted with EtOAc (6300 mL) and the solution washed with water, and dried over MgSO₄. Volatiles were removed from the solution in a rotary evaporator to yield compound 5.

To a solution of compound 5 in glacial acetic acid (1 mL) was added iron (50 mg) and refluxed for 30 minutes at 130° C. The reaction mixture was cooled to rt, volatiles removed in vacuo and extracted with EtOAc. The solution was washed with saturated aqueous NaHCO₃ solution, dried over MgSO₄, and volatiles removed in vacuo. The residue was separated by flash column chromatography to yield compound 6.

A slurry of compound 6 (27.5 mg) and compound 7 (13 mg) in acetic acid (0.5 mL) was heated to 100° C. with reflux condenser under nitrogen atmosphere for an hour and cooled to rt. Volatiles were removed in vacuo, the residue extracted with EtOH (3×4 mL). Volatiles fro the ethanolic solution were removed in a rotary evaporator and then in vacuo to yield compound 7.

Compound 7 (ca. 10 mg) was dissolved in acetone (5 mL), refluxed, and KMnO₄ was added until pink color persisted. The reaction mixture was filtered, excess KMnO₄ neutralised with sodium sulfite, and extracted with DCM. The DCM solution was dried over MgSO₄ and volatiles removed in a rotary evaporator to yield compound 8.

### Example 2

### Synthesis of Compound 11

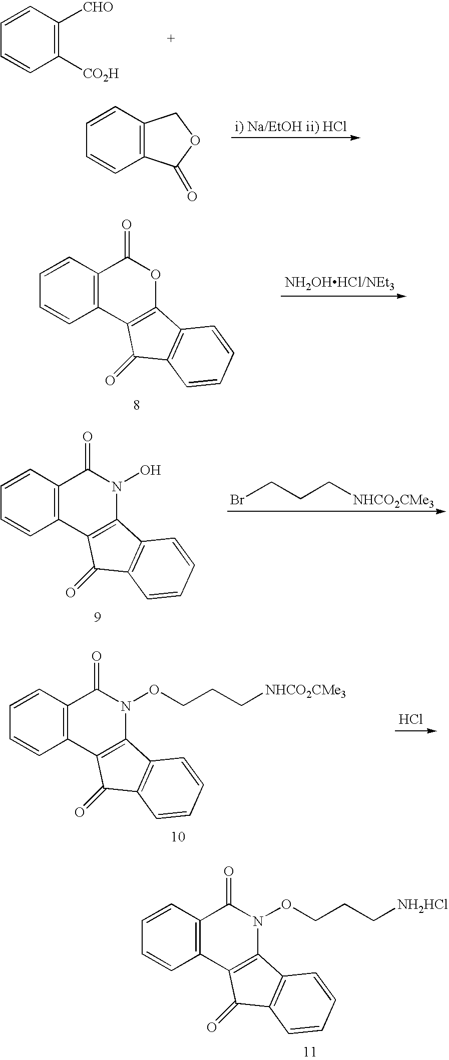

Sodium (1.84 g) was dissolved in Ethanol (absolute, 80 ml) under an N₂ atmosphere followed by the addition of 2-carboxybenzaldehyde (5.60 g) and phthalide (5.00 g). The mixture was refluxed for 1.5 h, water (80 mL) added to it, and volatiles removed in vacuo. Ice water (400 mL) was added to the residue, the aqueous portion washed with ether (2×100 mL), acidified (3M HCl) to pH 2, and extracted with ether (200 mL). The ether solution was dried over MgSO₄ and volatiles removed to yield a sticky pale yellow tar, slowly solidifying in high vacuo, which was mixed with conc. HCl (150 mL) and heated at 115° C. for 1.75 h. An orange precipitate was filtered off, washed with water, and volatiles removed in high vacuo to yield compound 8 which was characterized by ¹HNMR.

To a mixture of compound 8 and hydroxylamine hydrochloride (10.1 mg) in Chloroform (1.5 mL) was added triethylamine (20.2 mL), stirred at rt for 2.5 h. The reaction mixture was worked up using water-EtOAc and analyzed by TLC (CHCl₃/MeOH 9.5:05) to indicate completion of reaction. The reaction mixture was added to water (10 mL), and extracted with EtOAc. The EtOAc solution was dried over MgSO₄, volatiles evaporated, and the residual orange solid separated using flash column chromatography (Biotage, 0-25% MeOH/DCM) to yield compound 10 (14 mg) which was characterized by ¹HNMR.

A mixture of Compound 9 (14 mg), 3-Bromo-1-N-Bocpropylamine (18 mg), DMF (1.0 mL), and DIEA (0.05 mL), was stirred at rt overnight when a TLC (CHCl₃/MeOH 9.5:0:5) analysis showed completion of reaction. Water was added to the reaction and extracted with EtOAc. The crude product was separated by flash column chromatography (biotage, 0-15% MeOH/DCM to yield compound 11 which was characterized by ¹HNMR.

### Example 3

### Synthesis of Compound 14

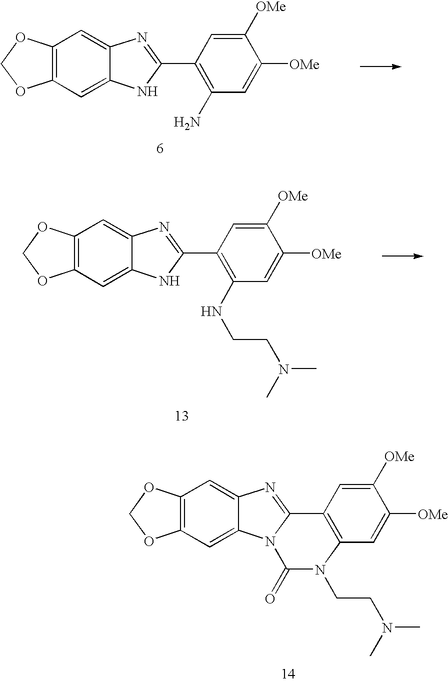

A mixture of Compound 6 (25 mg), N,N-dimethylaminoacetaldehyde hydrochloride (13 mg), methanol (1 mL) was heated at 65° C. for ca 2 h. NaBH₃CN (ca. 30 g) was added to the reaction mixture, stirred at rt overnight, at 50° C. for ca. 6 h and added to a solution of K₂CO₃ (300 mg) in water (15 mL), and extracted with EtOAC. The EtOAc solution was dried over MgSO₄ and volatiles removed to yield an yellow oil (compound 13, 37 mg) which was dissolved in triethylamine (TEA, 60 ml) and acetonitrile (1 ml) under N₂, the reaction mixture cooled in an ice bath and phosgene (20% in toluene, 25 mL) added to it. The temperature was raised to rt an stirred for 40 minutes. Mass spectra of the reaction mixture indicated that some compound 13 was present and phosgene (20% in toluene, 15 mL) was added to the reaction mixture and the heated at 50° C. After an hour, the reaction mixture was cooled to rt, water (10 mL) added to it and the precipitated tan solid collected by fine frit filtration to yield compound 14 which was characterized by ¹HNMR.

### Example 4

Cells are plated in 60-mm glass dishes (2×10⁵-1×10⁶ cells per dish) 1 to 2 days prior to compound testing. A solution of the compound of the invention to be tested is made immediately before the test and added to the cells in complete medium. Hypoxia (less than 200 ppm O₂) is achieved by exposing the glass dishes in pre-warmed, air tight aluminum jigs to a series of five rapid evacuations and flushings with 95% nitrogen plus 5% carbon dioxide in a 37° C. water bath on a shaking platform (controls are flushed as well). After the fifth evacuation and flushing, the platform (with water bath and jigs) is shaken for 5 minutes, then one more evacuation and flushing are performed, and the jigs are transferred to a shaker in a 37° C. incubator for the remainder of the 1 to 2 hour drug exposure. Levels of oxygenation between 200 ppm and air are achieved by varying the degree and number of evacuations. The oxygen concentrations in the medium and gas phases is checked using an oxygen electrode (Anima, Phoenixville, Pa.) in a specially modified aluminum jig that allows monitoring of both gas and liquid phases. Following the exposure to the drug, the aluminum vessels are opened, the drug washed off the cells by rinsing with medium, the cells trypsinized, and the cells are then plated for clonogenic survival in plastic Petri dishes. The plating efficiency of the cells is 60% or greater. Ten to 14 days later, the dishes are stained with crystal violet (0.25% in 95% ethanol), and colonies containing more than 50 cells are counted.

### Example 5

### Clonogenic Assay

The compounds of the invention are tested in the assay as follows. Exponentially growing human H460 cells (obtained from the ATCC) are seeded into 60 mm notched glass plates at a density of between 2.5 and 5×10⁵ cells per plate and grown in RPMI medium supplemented with 10% fetal bovine serum for 2 days prior to initiating drug treatment. On the day of the test, drug stocks of known concentrations are prepared in complete medium, and 2 ml of the desired stock added to each plate. To achieve complete equilibration between the surrounding gas phase and the liquid phase, the lid of the glass plate is removed and the plate shaken for 5 minutes on an orbital shaker. The plated is recovered and stored inside a glove-box. The glove-box was evacuated and gassed with either a certified anoxic gas mixture (95% nitrogen and 5% carbon dioxide) or with an aerobic (normoxic) gas mixture (95% air and 5% carbon dioxide). Cells are incubated with the drug for 2 hours at 37° C.

At the end of treatment with the test drug, plates are removed from each vessel, and the test drug is promptly removed from the cells. Plates are washed with phosphate buffered saline and a solution of trypsin-EDTA and trypsinized for 5 minutes at 37° C. Detached cells are neutralized with medium plus serum and collected by centrifugation for 5 min at 100×g. Cells are resuspended at approximately 1×10⁶ cells/ml and diluted 10 fold to yield stock concentrations for plating. The concentration of each stock is determined by counting with a Coulter Z2 particle counter. Known numbers of cells are plated, and the plates are placed in an incubator for between 7 and 10 days. Colonies are fixed and stained with a solution of 95% ethanol and 0.25% crystal violet. Colonies of greater than 50 cells are counted, and the surviving fraction is determined.

HT 29 and cell based clonogenic assays are performed in the same way as described above.

### Example 6

### Antiproliferation assay

To determine the effect of the compounds of the invention on cell proliferation, the antiproliferative activity of these compounds was tested in a multi-well Alamar Blue based assay (at 2 h and 3 days). Cell growth in the presence and absence of the test compound was compared, as measured by a fluorescence plate reader at excitation 550 nm and emission 590 nm (see Biosource International Inc., Tech Application Notes, Use of Alamar Blue in the measurement of Cell Viability and Toxicity, Determining IC₅₀). H460 cells (ATCC HTB-177 (NCI-H40), 4,000 cells/well/200 μl) were seeded in a 96 well plate in RPMI medium (Invitrogen Corporation, Carlsbad, Calif.). After 24 hours, these plates were divided into 3 groups—Control group, 2 h treatment group, and 3 day treatment group. A test compound was added to each plate in the treatment groups (2 h and 3 day) at various concentration (in 50 μl of medium). In the 2 h treatment group, after 2 h the cells were rinsed to remove the test compound and incubated for 3 days, followed by staining with AlamarBlue. The cells in the 3 day treatment group were incubated for 3 days, followed by staining with AlamarBlue. In the Control group, AlamarBlue was added to the plate at (i) day 0 and (ii) day 3 and measured to establish the control reading. In all the groups, the capacity of the cells to proliferate was measured 6 hours after addition of AlamarBlue by a fluorescence plate reader at excitation 550 nm and emission 590 nm and the 50% growth inhibitory concentration (GI₅₀ (also referred to IC₅₀ herein)) or the 90% growth inhibitory concentration (GI₉₀ (also referred to IC₉₀ herein)) of the topoisomerase inhibitors of the invention was calculated.

TABLE 1

| Compound No. (assay time period) | GI₅₀ (μM) | GI₉₀ (μM) |
|---|---|---|
| 11 (3 days) | 2 | 10 |
| 11 (2 h) | 10 | 32 |
| 14 (3 days) | 0.6 | 1 |

Under the experimental conditions used, GI₅₀ and GI₉₀ values for compounds 7 and 8 and were not determinable under the experimental condition.

### Example 7

### BrdU-TUNEL Assay

The effect of compound of the invention on apoptosis is determined as follows. PWR-1E cells (2×10⁵ cells/ml/well) are seeded in a 24 well plate. After 24 h the test compound is added at various concentrations. The culture media is removed after 24 h; the cells are rinsed with PBS buffer (200 μL) and incubated (5 min, 37° C.) with a solution of Guava Viacount CDR in PBS (1:3 v/v). Media (750 μL) containing at least 5% FBS is added to each well, the cells released by repeated pipeting, centrifuged, and the supernatant aspirated. The cells are resuspended in PBS buffer (150 μL) and fixed by incubating (60 min, 4° C.) with 4% paraformaldehyde in PBS. The cells are centrifuged, and the supernatant removed to a final volume of 15 μL. The cell pellets are resuspended, followed by dropwise addition of 200 μl of ice-cold ethanol (70%), and the cells are incubated at −20° C. at least for 2 hr. The cells are centrifuged, the supernatant is removed, washed, and is incubated with the DNA labeling mix (37° C., 60 min). The cells are washed, incubated (30 min) with anti-BrdU staining mix, washed again and analyzed on a Guava PCA-96 system (Guava Technologies, 25801 Industrial Boulevard, Hayward Calif. 94545-2991, USA).

The effect of the test compound on apoptosis of H460, PC3, CCRF, LNCaP, HT29 and MESSA cells is determined using the same protocol as described above.

### Example 8

### Cell Cycle Analysis

The effect of compound of the invention on the cell cycle is determined as follows. LNCaP cells (2×10⁵ cells/ml/well) are seeded in a 24 well plate. After 24 h, the test compound is added at various concentrations. The culture media is removed after 24 h, the cells are trypsinized, and centrifuged. The cell pellets are resuspended in 100 μl PBS buffer, after which 300 μl of ice-cold ethanol (96%) is added dropwise, and the cells are incubated at 4° C. for at least 24 hr. The cells are centrifuged and the supernatant is discarded. The cell cycle staining reagent (Guava Technologies, Hayward, Calif., USA, 200 μl) is added to each well. The cells are shielded from light and incubated at room temperature for 30 min. The samples are analyzed (Guava PCA-96 instrument, Cytosoft software, Guava Technologies, 25801 Industrial Boulevard, Hayward Calif. 94545-2991, USA).

The effect of the test compound on the cell cycle of H460, PC3, CCRF, HT29, MESSA and PWR-1E cells is determined using the same protocol as described above.

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and illustrative methods are for purposes of exemplification and not limitation of the following claims.

1. A compound selected from the group consisting of: 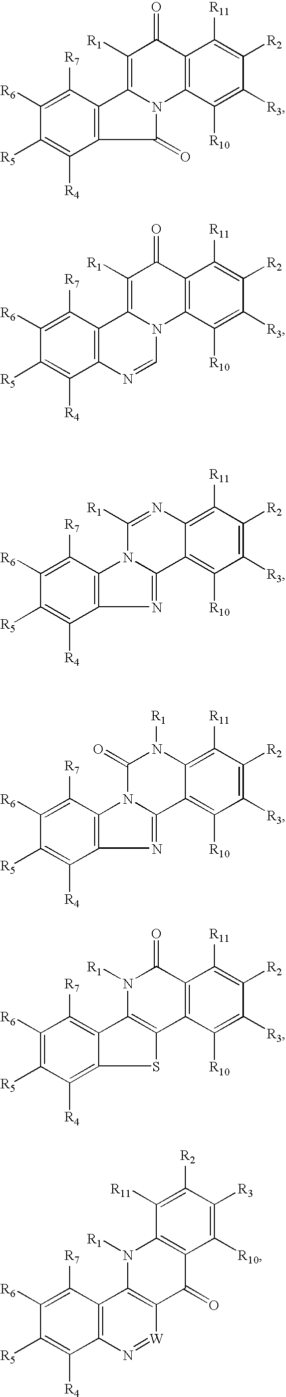 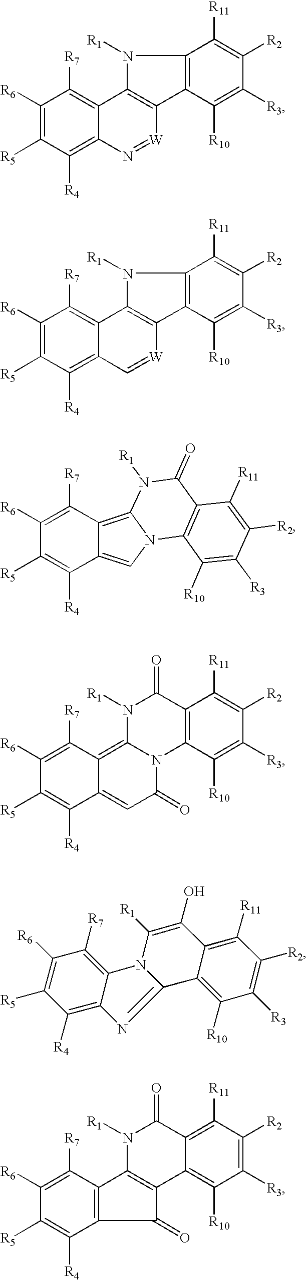 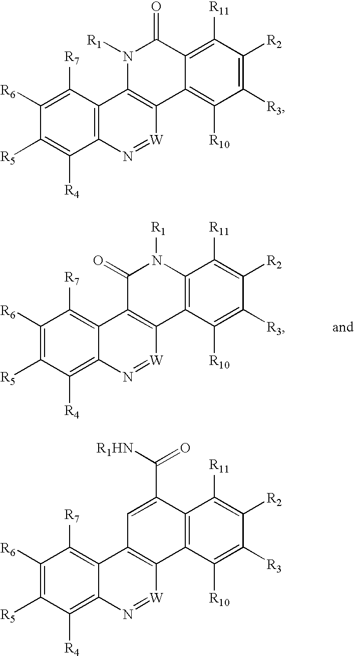
wherein
R₁ is C₁-C₆ alkyl, C₁-C₆ alkoxy, or C₁-C₆ alkylamino;
R₂ R₃, R₁₀, and R₁₁ are independently hydrogen, C₁-C₆ alkoxy, NO₂, amino, aminoalkyl, or hydroxyl;
R₄, R₅, R₆, and R₇ are independently hydrogen, C₁-C₆ alkoxy, NO₂, amino, aminoalkyl, or hydroxyl, or R₅ and R₆ together are —CH₂—O—CH₂—; and
W is —N═ or —CH═;
provided that for: 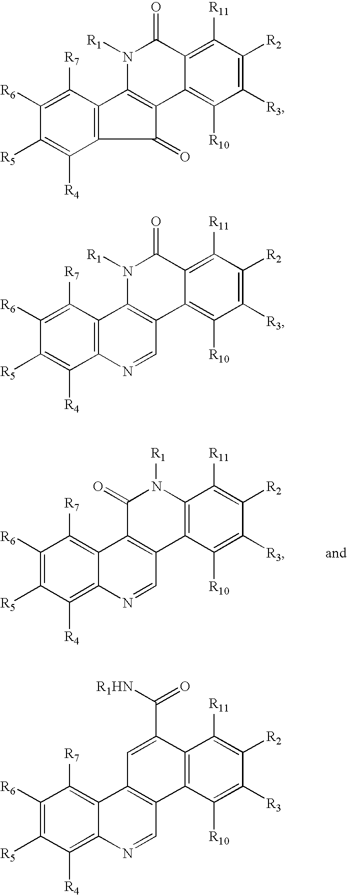
R₁ is not C₁-C₆ alkyl;
provided that for: 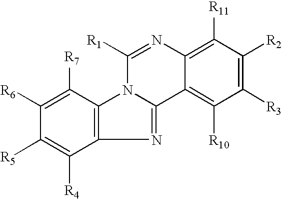
when R₅ and R₆ together are —O—CH₂—O—; R₄, R₇, R₁₀, and R₁₁ are hydrogen; and
R₂ and R₃ are —OMe; then R₁ is not —CH₂—CH₂—CH₂—NMe₂; and
provided that for: 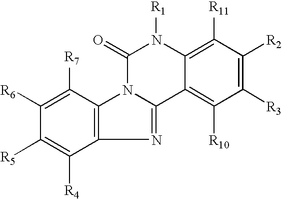
when R₅ and R₆ together are —O—CH₂—O—; R₄, R₇, R₁₀, and R₁ are hydrogen; and
R₂ and R₃ are —OMe; then R₁ is not —CH₂—CH₂—NMe₂.

2. The compound of claim 1 of formula

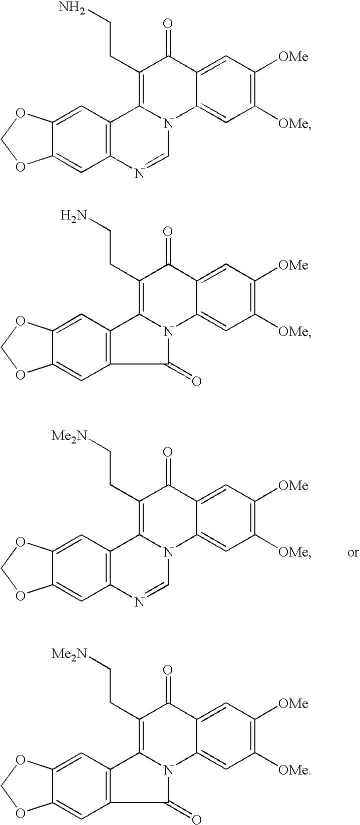

3. A compound selected from the group consisting of: 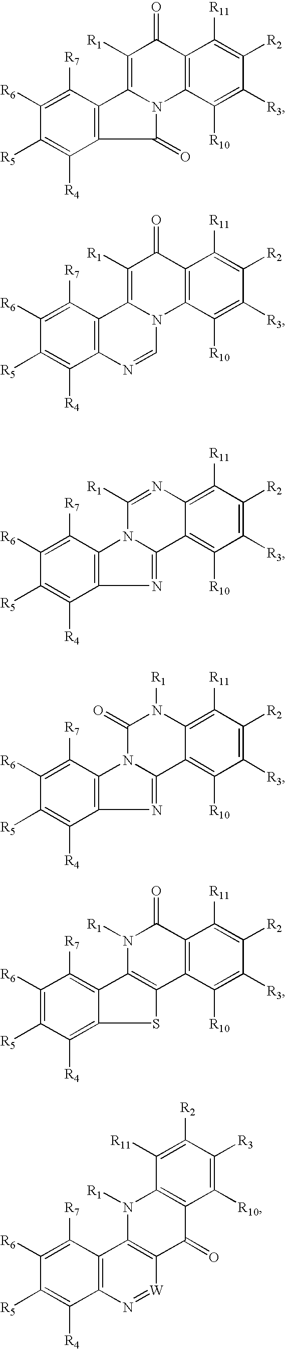 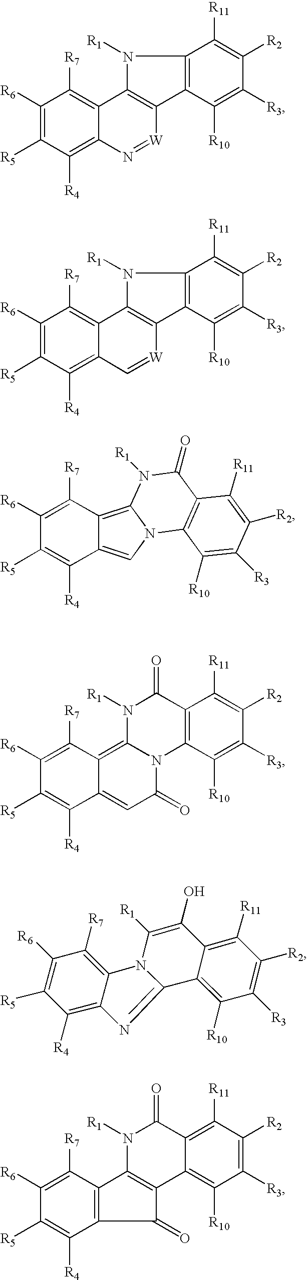 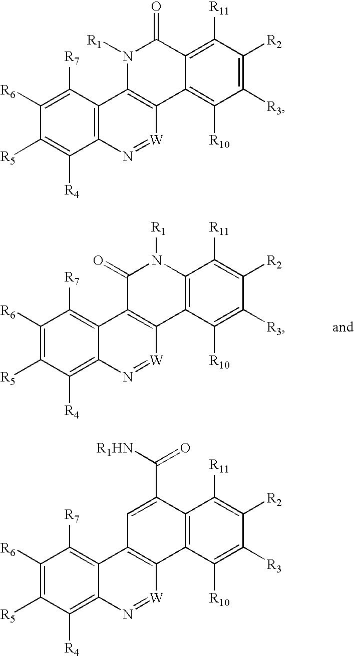
wherein
R₁ is C₁-C₆ alkyl, C₁-C₆ alkoxy, or C₁-C₆ alkylamino, R₈—[O—(C═O)]ₘ—NR₉—(CH₂)ₙ—, R₈—[O—(C═O)]ₘ—NR₉—(CH₂)ₙ—O—, R₈—[O—(C═O)]ₘ—NR₉—(CH₂)ₙ—NH, R₈—[O—(C═O)]ₘ—NR₉—, or R₈—[O—(C═O)]ₘ—NR₉—(CH₂)ₙ—N(R₈—[—(C═O)]ₘ)—;
R₂, R₃, R₁₀, and R₁₁ are independently hydrogen, C₁-C₆ alkoxy, NO₂, amino, aminoalkyl, hydroxyl, or R₈—[O—(C═O)]ₘ—NR₉—;
R₄, R₅, R₆, and R₇ are independently hydrogen, C₁-C₆ alkoxy, NO₂, amino, aminoalkyl, or hydroxyl, R₈—[O—(C═O)]ₘ—NR₉—; or R₅ and R₆ together are (—CH₂—O—CH₂—);
R₈ is a hypoxia labile protecting group, R₉ is hydrogen or C₁-C₆ alkyl; m is 0 or 1, n is from 1-6, and
W is —N═ or —CH═;
provided that if R₁ is C₁-C₆ alkyl, C₁-C₆ alkoxy, or C₁-C₆ alkylamino, then at least one of R₂-R₇, R₁₀, and R₁₁ is R₈—[O—(C═O)]ₘ—NR₉—;
provided that for 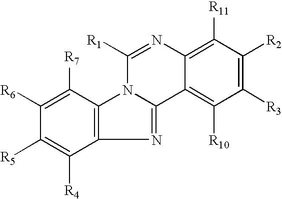
when R₅ and R₆ together are —O—CH₂—O—; R₄, R₇, R₁₀, and R₁₁ are hydrogen; and R₂ and R₃ are —OMe; then R₁ is not —CH₂—CH₂—CH₂—NMe-[(C═O)—O]—R₈; and
provided that for 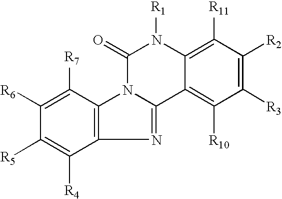
when R₅ and R₆ together are —O—CH₂—O—; R₄, R₇, R₁₀, and R₁₁ are hydrogen; and R₂ and R₃ are —OMe, then R₁ is not —CH₂—CH₂—NMe-[(C═O)—O]—R₈.

4. The compound of claim 3, wherein said compound is a hypoxia activated prodrug of a topoisomerase inhibitor.

5. A compound of claim 3 comprising at least one hypoxia labile protecting group.

6. A compound of claim 3 comprising at least two hypoxia labile protecting groups.

7. The compound of claim 3 wherein R₈ is selected from: 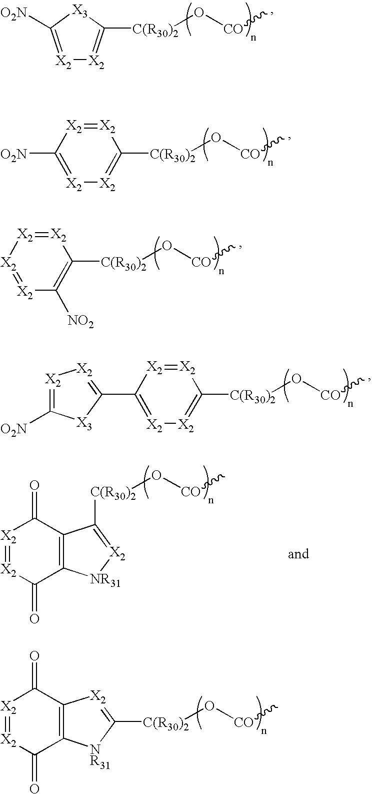
wherein each X₂ is N or CR₃₂;
X₃ is NR₃₁, S, or O;
each R₃₀ is independently hydrogen or alkyl;
R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;
R₃₂ is hydrogen, halogen, nitro, cyano, CO₂H, C₁-C₆ alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and
n=0, 1.

8. The compound of claim 7 wherein R₈ is selected from:

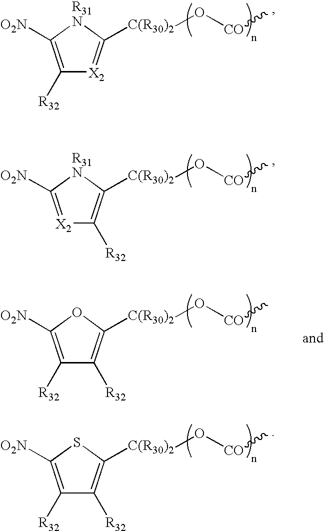

9. The compound of claim 5 having the formula

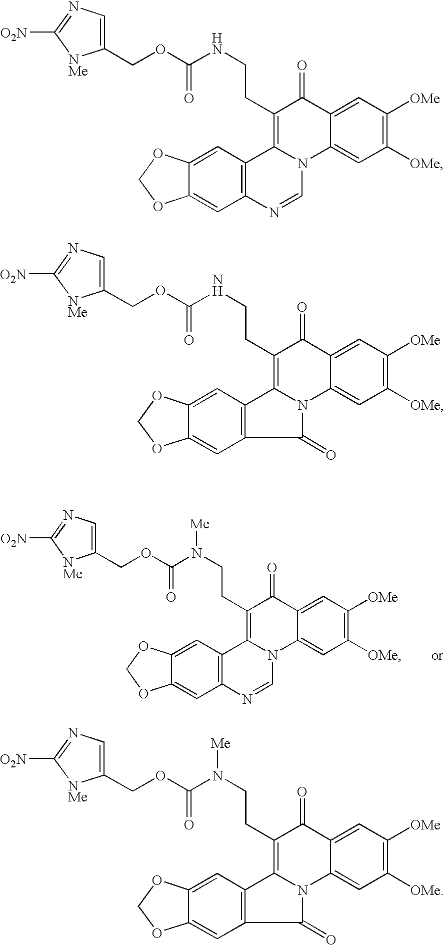

10. A pharmaceutical composition comprising a pharmaceutically acceptable carrier and a compound of claim 1.

11. A method of treating cancer comprising administering a therapeutically effective amount of a compound of claim 1 to a cancer patient.

12. A method of treating a hyperproliferative disease comprising administering a therapeutically effective amount of a compound of claim 1 to a patient having said disease.

13. The method of claim 12, wherein the hyperproliferative disease is selected from the group consisting of macular degeration, gout, psoriasis, rheumatoid arthritis, restenosis, benign prostatic hyperplasia, and multiple sclerosis.
